SEQUENCE LISTING (45 sequences)

SEQ ID NO: 1 - protein, 28 aa, Burkholderia ubonensis
GGDGSIAEYFNRPMHIHDWQIMDSGYYG

SEQ ID NO: 2 - DNA, 183 bp, Burkholderia ubonensis
atgaaaaatcgtagcaccaaagagagcttcgaaattacctgcattggcgatgtggatgtgattaccctgatgcaggatgcgagccgtgcgacaatgggaggcgatggcagcattgcggaatactttaaccgtccgatgcatattcatgattggcagattatggatagcggctattatggctga

SEQ ID NO: 3 - DNA, 675 bp, Burkholderia ubonensis
atgccgtatgcgctgagccagcatgcgcgtctggcgtgttatgaagatgatctgattattctgaccattcgtgataatcgttttcatctgatcaaagatgtgagccgtgatgcggtggacgcgttatatgaaccgatggcgggacagcgtggcgcaggactgcatgacgcgcttcgtattatgggcgtgctggaagagagtcgcgatcgtgcggatattccgcctgcgggactgcgtccgaaaagctatgtggaacagcgttggatgatgccgctgactcggcatgctccggcgaccttagtgggcaccgtggcgtcgctggtggcactgtatcgtgcaaccctgatgattaaactgggcggctttcgtcgtattgtgagcattggcaaatggccggctcgtatggcgagcggcagtgtcgatgtggatggcacagtgcaggctgcaatgggcgatctgaaccgtgtgtttgcgtgcgatgtgtctggcaatcgttgcctgacctatagcctggcgctgaccctgctgctgcgtcgtaaaattccgaatgtttcactggtggtgggcgtccgtacccgtccgttttttagccatgcgtgggttgaagtggacggtcgcgtggtgaatgataccgcggatctgcgtaaaaatctggcggtgatcctggaggtttga

SEQ ID NO: 4 - DNA, 1683 bp, Burkholderia ubonensis
atgttcattgcctaccctgagaacatagcgaagcatttggaatacatcattgatgaatgggctggtcgtaatcgtctgaccacccacctgcatggtaagttcgtggtgcgttgtagcgatcgttggaccgtgagcaagtggggcaatttcattgagttctttgaagggatggcttatgagtggccgacctgccaggcttggccagcgccggagcgtcgtgctcgtcttagtaatagcattggctattttaccagcattctgttggattccgataccctggaagtcgtgcgcagcctgtatcgtgcgaccgacatcttttataccgaaagcgatggcatgatgttagcgtgtagtgaactggctgtggtggtcgcgcttcgtggcggatttgcacgccagcgtattgatgtggattattgccatgatttcatagcacaccaacaaaagttcgacggacatacgtcatttgaatcaattaacgaggtgatgctgggcgaatgtatacggatgagcacaagcgatatcatttcagctgcgtttgtgaatcgtccgattgtcccgagcggcgacatcgtggacaccttgcgtgataccttagcggcatttacacgtccatttgatggcaccgtcctgatgtttagcgggggtctggatagcagtaccctgttgtggactctgctggaatctggcactaaaccgctggtgctgcacagcgagtctgggccggatgcgcgtgacagcgaataccaggacgcagcggcagtggcactggatctgggctgcgaaattcagcgtttcgtgccgggacgcgaggactatagccgcgcttttactatcagtgatgacggccaaagcagcagtccgtatgatattcccatcttcctgtctcgtagctctgctcgttcgggcttatctatcgatgaaaccagcctgctggtgaccgggcatgggggtgaccatgtgttcgtgcagaaccccgaaaacaactcctgcttggcggctcttcaagcgggacgggtgtttgagtatctgcgtacggtgcgtaaactgagccgtctgaaaggccgtcggggcgtggagattgtacggcataacctgcgcctgcttatgggaggccatctgctgtcaggttcgttcccggattggctgccgcgtccgcggcaccgctccgcacgtcggacaggccactacttaattcgcgacctggaccggcgtatggcgaaacatactcatctgagcgccattctgcaggctttacagagcgcaagcattccgcgcaacggaccgcccatgttggcgccgttgttgctgcaaaatgtgattggccatatgatgggcataccggttcaggatacgtttactgaaacccatgatcgtgtgacacttcgtgagtcgatttatcgtcagtctggcaaatcttttgcgtggcgtcgtaccaaacgtgcgtccagcgctttcctgtttgaactgttgagccagtccgaagtgaatttagccgatctgatcgaccgcagccactttgttccactgttgcatattgatcgtcgggcattactggcggaagtgcgtcagaattgccggatagcgctgaccggcaactttaaacatattgtcaacttgtataagattgaagcgcaccttcgctcaatagagcatcagtctgcagaactaaccagaccatga

SEQ ID NO: 5 - DNA, 1749 bp, Burkholderia ubonensis
atgaagcgttggatcggtatctattctgagatcggccaccatttgcaacgccaggaacggtactttgtagtagcaattctgttttgcacccttggtgctgcggccagcatggcgatgagcccggtgtttttagggcgtctggcggattcactgcttgcggcggatcgtcgtatgcccgcgtacattatctacttagcggcaagctatttgatcaccattgctatgccaaagctgctgggcaccgtagatctgtacctgcagtcaatgttgcgtttacgtgcgaaccgtagcctgttagccgggtacttcaactatctgtgtcggcaacccgagagttttttcgtgaataagaatagtggtgagcttacccaagagatcacccaagcgtctaatgatctttacctgattgtacggaacctgaccactagccttatctcgccgattgtgcaggtgagcattgcggtggtcgtccttgcgagcaatcatgacctgttggtggcggggacgatagcgatttatgtggctttgttcgtaacaaacaatgtaatacatggccgtcgtttggtagaactgaaattccgttgcatggatgcaggtcggaaaagctatggaacgttgacggacagcatcaccaatattcaggtggcgcgtcagtttaatgggtatcgtttcctgttgagccgctatcaacgggtgcttgacgaagaccgtcatacacagggcgactactggaagatctctctgcgtatgcagtttttcaacgcgtgtctgtttgtgggcctgtttggcgtaacctttctgatggcgctgcacgaagtagtgaccggtgcgcgctctattggcaatttcgtgcttgtcgccgcgtataccgtgaccctgttaagccccatcgagattctgggcaatatgtttaccgaaattaaccagagtctggtgacctttgggcgttttcttgataaattgtcagcagccacagctcctcttagccagcgtgcgcctaagccggcagttaagtccgcggcaccggctatcgaatttgaacgtgtgtgcgtgacctatccgggtgccaatcgccaggcattaactgatgtgggctttacagtggatgccggaaagcgtgtagcgataactggtccctctggagcaggcaagagcagcctggtgaaagttctgacccgccaacttgtggcggaagaaggagccattcgtatttttggcgaggatatcttatgtattgatgcgcagaccctgagcgaacgtattggctgcgtgtcacaggacgtactgctgtttaaagataccttacggtttaacttgcagatttcgcgccctgatgcttcggacgcggacatggtcactgcacttgagtgcgcgggactgacggatcttttagtggacttacctgccgggttggacacgatgttaggcgatcgtggcgcaacactttctggaggtcagcgtcagcggttggcgttagcccgcttgttcctgcgtgcccccgacattgtgttggttgatgaaggcacctcttcgctggatttggtaactgagcagtatgttctggacaaggtgtttgaagtgtttagcgacaaaaccatagtgatgataacccaccgtcctagcgcgatgaccaaagttgatgccgtgattatcatgagcgatggtcgtattgacgatcatgcggaaccggatgtgcttcgtagccgtaataccttttttgcgcgtgttgtggaatcttctttgcgttga

SEQ ID NO: 6 - DNA, 45 bp, Bacteriophage T5
tcataaaaaatttatttgctttgtgagcggataacaattataata

SEQ ID NO: 7 - DNA, 96 bp, Escherichia coli
catcaattaagaaaaaaatttagcttgtagataaattcagaagttttattattccaattgagtgtaaaggcataactacaggagggagtgtgcaaa

SEQ ID NO: 8 - DNA, 4618 bp, Artificial Sequence
  recombinant vector
tcataaaaaatttatttgctttgtgagcggataacaattataatagattcaattgtgagcggataacaatttcacacagaattcattaaagaggagaaattaactatgaaaaatcgtagcaccaaagagagcttcgaaattacctgcattggcgatgtggatgtgattaccctgatgcaggatgcgagccgtgcgacaatgggaggcgatggcagcattgcggaatactttaaccgtccgatgcatattcatgattggcagattatggatagcggctattatggctgaaagcttaattagctgagcttggactcctgttgatagatccagtaatgacctcagaactccatctggatttgttcagaacgctcggttgccgccgggcgttttttattggtgagaatccaagctagccatcaattaagaaaaaaatttagcttgtagataaattcagaagttttattattccaattgagtgtaaaggcataactacaggagggagtgtgcaaaatgccgtatgcgctgagccagcatgcgcgtctggcgtgttatgaagatgatctgattattctgaccattcgtgataatcgttttcatctgatcaaagatgtgagccgtgatgcggtggacgcgttatatgaaccgatggcgggacagcgtggcgcaggactgcatgacgcgcttcgtattatgggcgtgctggaagagagtcgcgatcgtgcggatattccgcctgcgggactgcgtccgaaaagctatgtggaacagcgttggatgatgccgctgactcggcatgctccggcgaccttagtgggcaccgtggcgtcgctggtggcactgtatcgtgcaaccctgatgattaaactgggcggctttcgtcgtattgtgagcattggcaaatggccggctcgtatggcgagcggcagtgtcgatgtggatggcacagtgcaggctgcaatgggcgatctgaaccgtgtgtttgcgtgcgatgtgtctggcaatcgttgcctgacctatagcctggcgctgaccctgctgctgcgtcgtaaaattccgaatgtttcactggtggtgggcgtccgtacccgtccgttttttagccatgcgtgggttgaagtggacggtcgcgtggtgaatgataccgcggatctgcgtaaaaatctggcggtgatcctggaggtttgatgttcattgcctaccctgagaacatagcgaagcatttggaatacatcattgatgaatgggctggtcgtaatcgtctgaccacccacctgcatggtaagttcgtggtgcgttgtagcgatcgttggaccgtgagcaagtggggcaatttcattgagttctttgaagggatggcttatgagtggccgacctgccaggcttggccagcgccggagcgtcgtgctcgtcttagtaatagcattggctattttaccagcattctgttggattccgataccctggaagtcgtgcgcagcctgtatcgtgcgaccgacatcttttataccgaaagcgatggcatgatgttagcgtgtagtgaactggctgtggtggtcgcgcttcgtggcggatttgcacgccagcgtattgatgtggattattgccatgatttcatagcacaccaacaaaagttcgacggacatacgtcatttgaatcaattaacgaggtgatgctgggcgaatgtatacggatgagcacaagcgatatcatttcagctgcgtttgtgaatcgtccgattgtcccgagcggcgacatcgtggacaccttgcgtgataccttagcggcatttacacgtccatttgatggcaccgtcctgatgtttagcgggggtctggatagcagtaccctgttgtggactctgctggaatctggcactaaaccgctggtgctgcacagcgagtctgggccggatgcgcgtgacagcgaataccaggacgcagcggcagtggcactggatctgggctgcgaaattcagcgtttcgtgccgggacgcgaggactatagccgcgcttttactatcagtgatgacggccaaagcagcagtccgtatgatattcccatcttcctgtctcgtagctctgctcgttcgggcttatctatcgatgaaaccagcctgctggtgaccgggcatgggggtgaccatgtgttcgtgcagaaccccgaaaacaactcctgcttggcggctcttcaagcgggacgggtgtttgagtatctgcgtacggtgcgtaaactgagccgtctgaaaggccgtcggggcgtggagattgtacggcataacctgcgcctgcttatgggaggccatctgctgtcaggttcgttcccggattggctgccgcgtccgcggcaccgctccgcacgtcggacaggccactacttaattcgcgacctggaccggcgtatggcgaaacatactcatctgagcgccattctgcaggctttacagagcgcaagcattccgcgcaacggaccgcccatgttggcgccgttgttgctgcaaaatgtgattggccatatgatgggcataccggttcaggatacgtttactgaaacccatgatcgtgtgacacttcgtgagtcgatttatcgtcagtctggcaaatcttttgcgtggcgtcgtaccaaacgtgcgtccagcgctttcctgtttgaactgttgagccagtccgaagtgaatttagccgatctgatcgaccgcagccactttgttccactgttgcatattgatcgtcgggcattactggcggaagtgcgtcagaattgccggatagcgctgaccggcaactttaaacatattgtcaacttgtataagattgaagcgcaccttcgctcaatagagcatcagtctgcagaactaaccagaccatgaagcgttggatcggtatctattctgagatcggccaccatttgcaacgccaggaacggtactttgtagtagcaattctgttttgcacccttggtgctgcggccagcatggcgatgagcccggtgtttttagggcgtctggcggattcactgcttgcggcggatcgtcgtatgcccgcgtacattatctacttagcggcaagctatttgatcaccattgctatgccaaagctgctgggcaccgtagatctgtacctgcagtcaatgttgcgtttacgtgcgaaccgtagcctgttagccgggtacttcaactatctgtgtcggcaacccgagagttttttcgtgaataagaatagtggtgagcttacccaagagatcacccaagcgtctaatgatctttacctgattgtacggaacctgaccactagccttatctcgccgattgtgcaggtgagcattgcggtggtcgtccttgcgagcaatcatgacctgttggtggcggggacgatagcgatttatgtggctttgttcgtaacaaacaatgtaatacatggccgtcgtttggtagaactgaaattccgttgcatggatgcaggtcggaaaagctatggaacgttgacggacagcatcaccaatattcaggtggcgcgtcagtttaatgggtatcgtttcctgttgagccgctatcaacgggtgcttgacgaagaccgtcatacacagggcgactactggaagatctctctgcgtatgcagtttttcaacgcgtgtctgtttgtgggcctgtttggcgtaacctttctgatggcgctgcacgaagtagtgaccggtgcgcgctctattggcaatttcgtgcttgtcgccgcgtataccgtgaccctgttaagccccatcgagattctgggcaatatgtttaccgaaattaaccagagtctggtgacctttgggcgttttcttgataaattgtcagcagccacagctcctcttagccagcgtgcgcctaagccggcagttaagtccgcggcaccggctatcgaatttgaacgtgtgtgcgtgacctatccgggtgccaatcgccaggcattaactgatgtgggctttacagtggatgccggaaagcgtgtagcgataactggtccctctggagcaggcaagagcagcctggtgaaagttctgacccgccaacttgtggcggaagaaggagccattcgtatttttggcgaggatatcttatgtattgatgcgcagaccctgagcgaacgtattggctgcgtgtcacaggacgtactgctgtttaaagataccttacggtttaacttgcagatttcgcgccctgatgcttcggacgcggacatggtcactgcacttgagtgcgcgggactgacggatcttttagtggacttacctgccgggttggacacgatgttaggcgatcgtggcgcaacactttctggaggtcagcgtcagcggttggcgttagcccgcttgttcctgcgtgcccccgacattgtgttggttgatgaaggcacctcttcgctggatttggtaactgagcagtatgttctggacaaggtgtttgaagtgtttagcgacaaaaccatagtgatgataacccaccgtcctagcgcgatgaccaaagttgatgccgtgattatcatgagcgatggtcgtattgacgatcatgcggaaccggatgtgcttcgtagccgtaataccttttttgcgcgtgttgtggaatcttctttgcgttgaccatgg

SEQ ID NO: 9 - DNA, 1478 bp, Artificial Sequence
  gBlock 1
gcgttttttattggtgagaatccaagctagccatcaattaagaaaaaaatttagcttgtagataaattcagaagttttattattccaattgagtgtaaaggcataactacaggagggagtgtgcaaaatgccgtatgcgctgagccagcatgcgcgtctggcgtgttatgaagatgatctgattattctgaccattcgtgataatcgttttcatctgatcaaagatgtgagccgtgatgcggtggacgcgttatatgaaccgatggcgggacagcgtggcgcaggactgcatgacgcgcttcgtattatgggcgtgctggaagagagtcgcgatcgtgcggatattccgcctgcgggactgcgtccgaaaagctatgtggaacagcgttggatgatgccgctgactcggcatgctccggcgaccttagtgggcaccgtggcgtcgctggtggcactgtatcgtgcaaccctgatgattaaactgggcggctttcgtcgtattgtgagcattggcaaatggccggctcgtatggcgagcggcagtgtcgatgtggatggcacagtgcaggctgcaatgggcgatctgaaccgtgtgtttgcgtgcgatgtgtctggcaatcgttgcctgacctatagcctggcgctgaccctgctgctgcgtcgtaaaattccgaatgtttcactggtggtgggcgtccgtacccgtccgttttttagccatgcgtgggttgaagtggacggtcgcgtggtgaatgataccgcggatctgcgtaaaaatctggcggtgatcctggaggtttgatgttcattgcctaccctgagaacatagcgaagcatttggaatacatcattgatgaatgggctggtcgtaatcgtctgaccacccacctgcatggtaagttcgtggtgcgttgtagcgatcgttggaccgtgagcaagtggggcaatttcattgagttctttgaagggatggcttatgagtggccgacctgccaggcttggccagcgccggagcgtcgtgctcgtcttagtaatagcattggctattttaccagcattctgttggattccgataccctggaagtcgtgcgcagcctgtatcgtgcgaccgacatcttttataccgaaagcgatggcatgatgttagcgtgtagtgaactggctgtggtggtcgcgcttcgtggcggatttgcacgccagcgtattgatgtggattattgccatgatttcatagcacaccaacaaaagttcgacggacatacgtcatttgaatcaattaacgaggtgatgctgggcgaatgtatacggatgagcacaagcgatatcatttcagctgcgtttgtgaatcgtccgattgtcccgagcggcgacatcgtggacaccttgcgtgataccttagcggcatttacacgtccatttgatggcaccgtcctgatgtttagcgggggtctggatagcagtaccctgttgtggactct

SEQ ID NO: 10 - DNA, 1484 bp, Artificial Sequence
  gBlock 2
ggatagcagtaccctgttgtggactctgctggaatctggcactaaaccgctggtgctgcacagcgagtctgggccggatgcgcgtgacagcgaataccaggacgcagcggcagtggcactggatctgggctgcgaaattcagcgtttcgtgccgggacgcgaggactatagccgcgcttttactatcagtgatgacggccaaagcagcagtccgtatgatattcccatcttcctgtctcgtagctctgctcgttcgggcttatctatcgatgaaaccagcctgctggtgaccgggcatgggggtgaccatgtgttcgtgcagaaccccgaaaacaactcctgcttggcggctcttcaagcgggacgggtgtttgagtatctgcgtacggtgcgtaaactgagccgtctgaaaggccgtcggggcgtggagattgtacggcataacctgcgcctgcttatgggaggccatctgctgtcaggttcgttcccggattggctgccgcgtccgcggcaccgctccgcacgtcggacaggccactacttaattcgcgacctggaccggcgtatggcgaaacatactcatctgagcgccattctgcaggctttacagagcgcaagcattccgcgcaacggaccgcccatgttggcgccgttgttgctgcaaaatgtgattggccatatgatgggcataccggttcaggatacgtttactgaaacccatgatcgtgtgacacttcgtgagtcgatttatcgtcagtctggcaaatcttttgcgtggcgtcgtaccaaacgtgcgtccagcgctttcctgtttgaactgttgagccagtccgaagtgaatttagccgatctgatcgaccgcagccactttgttccactgttgcatattgatcgtcgggcattactggcggaagtgcgtcagaattgccggatagcgctgaccggcaactttaaacatattgtcaacttgtataagattgaagcgcaccttcgctcaatagagcatcagtctgcagaactaaccagaccatgaagcgttggatcggtatctattctgagatcggccaccatttgcaacgccaggaacggtactttgtagtagcaattctgttttgcacccttggtgctgcggccagcatggcgatgagcccggtgtttttagggcgtctggcggattcactgcttgcggcggatcgtcgtatgcccgcgtacattatctacttagcggcaagctatttgatcaccattgctatgccaaagctgctgggcaccgtagatctgtacctgcagtcaatgttgcgtttacgtgcgaaccgtagcctgttagccgggtacttcaactatctgtgtcggcaacccgagagttttttcgtgaataagaatagtggtgagcttacccaagagatcacccaagcgtctaatgatctttacctgattgtacggaacctgaccactagccttatctcgccgattgtgcaggtgag

SEQ ID NO: 11 - DNA, 1349 bp, Artificial Sequence
  gBlock 3
cttatctcgccgattgtgcaggtgagcattgcggtggtcgtccttgcgagcaatcatgacctgttggtggcggggacgatagcgatttatgtggctttgttcgtaacaaacaatgtaatacatggccgtcgtttggtagaactgaaattccgttgcatggatgcaggtcggaaaagctatggaacgttgacggacagcatcaccaatattcaggtggcgcgtcagtttaatgggtatcgtttcctgttgagccgctatcaacgggtgcttgacgaagaccgtcatacacagggcgactactggaagatctctctgcgtatgcagtttttcaacgcgtgtctgtttgtgggcctgtttggcgtaacctttctgatggcgctgcacgaagtagtgaccggtgcgcgctctattggcaatttcgtgcttgtcgccgcgtataccgtgaccctgttaagccccatcgagattctgggcaatatgtttaccgaaattaaccagagtctggtgacctttgggcgttttcttgataaattgtcagcagccacagctcctcttagccagcgtgcgcctaagccggcagttaagtccgcggcaccggctatcgaatttgaacgtgtgtgcgtgacctatccgggtgccaatcgccaggcattaactgatgtgggctttacagtggatgccggaaagcgtgtagcgataactggtccctctggagcaggcaagagcagcctggtgaaagttctgacccgccaacttgtggcggaagaaggagccattcgtatttttggcgaggatatcttatgtattgatgcgcagaccctgagcgaacgtattggctgcgtgtcacaggacgtactgctgtttaaagataccttacggtttaacttgcagatttcgcgccctgatgcttcggacgcggacatggtcactgcacttgagtgcgcgggactgacggatcttttagtggacttacctgccgggttggacacgatgttaggcgatcgtggcgcaacactttctggaggtcagcgtcagcggttggcgttagcccgcttgttcctgcgtgcccccgacattgtgttggttgatgaaggcacctcttcgctggatttggtaactgagcagtatgttctggacaaggtgtttgaagtgtttagcgacaaaaccatagtgatgataacccaccgtcctagcgcgatgaccaaagttgatgccgtgattatcatgagcgatggtcgtattgacgatcatgcggaaccggatgtgcttcgtagccgtaataccttttttgcgcgtgttgtggaatcttctttgcgttgaccatgggcaaatattatacgcaaggcgac

SEQ ID NO: 12 - DNA, 55 bp, Artificial Sequence
  bubA Asmbly F1
aatagcgaattcattaaagaggagaaattaactatgaaaaatcgtagcaccaaag

SEQ ID NO: 13 - DNA, 56 bp, Artificial Sequence
  bubA Asmbly R2
cacatcgccaatgcaggtaatttcgaagctctctttggtgctacgatttttcatag

SEQ ID NO: 14 - DNA, 56 bp, Artificial Sequence
  bubA Asmbly F3
acctgcattggcgatgtggatgtgattaccctgatgcaggatgcgagccgtgcgac

SEQ ID NO: 15 - DNA, 56 bp, Artificial Sequence
  bubA Asmbly R4
gacggttaaagtattccgcaatgctgccatcgcctcccattgtcgcacggctcgca

SEQ ID NO: 16 - DNA, 56 bp, Artificial Sequence
  bubA Asmbly F5
attgcggaatactttaaccgtccgatgcatattcatgattggcagattatggatag

SEQ ID NO: 17 - DNA, 52 bp, Artificial Sequence
  bubA Asmbly R6
aggtcaagctttcagccataatagccgctatccataatctgccaatcatgaa

SEQ ID NO: 18 - DNA, 20 bp, Artificial Sequence
  bub cluster SeqF1
cagtgtcgatgtggatggca

SEQ ID NO: 19 - DNA, 23 bp, Artificial Sequence
  bub cluster SeqF2
caacaaaagttcgacggacatac

SEQ ID NO: 20 - DNA, 20 bp, Artificial Sequence
  bub cluster SeqF3
tcaggttcgttcccggattg

SEQ ID NO: 21 - DNA, 20 bp, Artificial Sequence
  bub cluster SeqF4
gatcgtcgtatgcccgcgta

SEQ ID NO: 22 - DNA, 20 bp, Artificial Sequence
  bub cluster SeqF5
gctctattggcaatttcgtg

SEQ ID NO: 23 - DNA, 47 bp, Artificial Sequence
  uboA-EcoRI-For
aatagcgaattcattaaagaggagaaattaactatgaaaaatcgtag

SEQ ID NO: 24 - DNA, 29 bp, Artificial Sequence
  pQE-HindIII-Rev
caacaggagtccaagctcagctaattaag

SEQ ID NO: 25 - DNA, 28 bp, Artificial Sequence
  uboA 16L-For
gatggcagcctggcggaatactttaacc

SEQ ID NO: 26 - DNA, 28 bp, Artificial Sequence
  ubo 16L-Rev
ggttaaagtattccgccaggctgccatc

SEQ ID NO: 27 - DNA, 31 bp, Artificial Sequence
  uboA P13C For
gaatactttaaccgttgcatgcatattcatg

SEQ ID NO: 28 - DNA, 31 bp, Artificial Sequence
  uboA P13C Rev
catgaatatgcatgcaacggttaaagtattc

SEQ ID NO: 29 - DNA, 31 bp, Artificial Sequence
  uboA M14C For
tactttaaccgtccgtgccatattcatgatt

SEQ ID NO: 30 - DNA, 31 bp, Artificial Sequence
  uboA M14C Rev
aatcatgaatatggcacggacggttaaagta

SEQ ID NO: 31 - DNA, 31 bp, Artificial Sequence
  uboA H15A-For
ctttaaccgtccgatggcgattcatgattgg

SEQ ID NO: 32 - DNA, 31 bp, Artificial Sequence
  uboA H15A-Rev
ccaatcatgaatcgccatcggacggttaaag

SEQ ID NO: 33 - DNA, 26 bp, Artificial Sequence
  uboA H17A-For
gtccgatgcatattgcggattggcag

SEQ ID NO: 34 - DNA, 26 bp, Artificial Sequence
  uboA-H17A-Rev
ctgccaatccgcaatatgcatcggac

SEQ ID NO: 35 - DNA, 29 bp, Artificial Sequence
  uboA D18N For
cgatgcatattcataattggcagattatg

SEQ ID NO: 36 - DNA, 29 bp, Artificial Sequence
  uboA D18N Rev
cataatctgccaattatgaatatgcatcg

SEQ ID NO: 37 - DNA, 41 bp, Artificial Sequence
  uboA D23N Rev
acattaagctttcagccataatagccgctattcataatctg

SEQ ID NO: 38 - DNA, 41 bp, Artificial Sequence
  uboA S24A Rev
tgcttaagctttcagccataatagccggcatccataatctg

SEQ ID NO: 39 - DNA, 31 bp, Artificial Sequence
  uboA W19C For
atgcatattcatgattgccagattatggata

SEQ ID NO: 40 - DNA, 31 bp, Artificial Sequence
  uboA W19C Rev
tatccataatctggcaatcatgaatatgcat

SEQ ID NO: 41 - DNA, 47 bp, Artificial Sequence
  ubo I21L Rev
gattggcagctgatggatagcggctattatggctgaaagcttaagaa

SEQ ID NO: 42 - DNA, 28 bp, Artificial Sequence
  uboA G28C Rev
taattaagctttcagcaataatagccgc

SEQ ID NO: 43 - DNA, 32 bp, Artificial Sequence
  uboA Y26F Rev
taattaagctttcagccataaaagccgctatc

SEQ ID NO: 44 - DNA, 29 bp, Artificial Sequence
  uboA Y27F Rev
taattaagctttcagccaaaatagccgct

SEQ ID NO: 45 - protein, 20 aa, Burkholderia ubonensis
GSIAEYFNRPMHIHDWQIMD

(19) United States
(12) Patent Application Publication
(10) Pub. No.: US 2022/0380415 A1
(43) Pub. Date: Dec. 1, 2022

(54) Antibiotic Peptides, Compositions and Uses Thereof

(71) Applicant: The Trustees of Princeton University, Princeton, NJ (US)

(72) Inventors: A. James Link, Belle Mead, NJ (US); Wai Ling Cheung-Lee, Voorhees, NJ (US)

(21) Appl. No.: 17/597,987

(22) PCT Filed: Aug. 7, 2020
(86) PCT No.: PCT/US2020/045413

§ 371 Date: Jan. 31, 2022

Related U.S. Application Data
(60) Provisional application No. 62/883,955, filed on Aug. 7, 2019.

(51) Int. Cl. C07K 14/195; A61P 31/04

(52) U.S. Cl. CPC C07K 14/195; A61P 31/04; A61K 38/00

(57) ABSTRACT

The present disclosure provides ubonodin peptides, pharmaceutical formulations comprising ubonodin peptides and nucleic acids encoding ubonodin peptides. The disclosure also provides methods of treating Burkholderia infections in a subject in need thereof utilizing the described ubonodin peptides and pharmaceutical formulations.

### RELATED APPLICATION(S)

This application is the U.S. National Stage of International Application No. PCT/US2020/045413, filed Aug. 7, 2020, published in English, which claims the benefit of U.S. Provisional Application No. 62/883,955, filed on Aug. 7, 2019. The entire teachings of the above applications are incorporated herein by reference.

### GOVERNMENT SUPPORT

This invention was made with government support under Grant No. GM107036 awarded by the National Institutes of Health. The government has certain rights in the invention.

### INCORPORATION BY REFERENCE OF MATERIAL IN ASCII TEXT FILE

This application incorporates by reference the Sequence Listing contained in the following ASCII text file:

a) File name: 53911024002_Sequence_Listing.txt; created Jan. 31, 2022, 25,780 Bytes in size.

### BACKGROUND

Burkholderia is a genus of Gram-negative Proteobacteria comprised of resilient and ubiquitous bacteria that are mainly environmental saprophytes.¹ Many of its members though, are opportunistic pathogens that can cause fatal diseases. Burkholderia mallei and Burkholderia pseudomallei, are classified as Tier 1 Select Agents by the US Federal Select Agent Program, causing glanders in animals and melioidosis in humans respectively.¹⁻² The Burkholderia cepacia complex (Bcc) consists of more than 20 closely related species of which many are opportunistic plant and human pathogens.^{1, 3-4} Bcc members are especially dangerous to patients with an underlying lung disease, such as those with cystic fibrosis (CF), causing deadly pneumonia. Bcc infections are difficult to treat due to their innate resistance to many antibiotics, their ability to persist even with aggressive antibiotic treatment, and their ability to acquire resistance to these antibiotics.^{1, 3-5}

### SUMMARY

In one aspect, the present invention provides isolated ubonodin peptides comprising an amino acid sequence having at least 70% sequence identity to the sequence of SEQ ID NO: 1, provided that the peptide does not consist of SEQ ID NO:1. In some embodiments, the ubonodin peptide comprises an amino acid sequence having at least 75%, at least 80%, at least 85%, at least 90% or at least 95% sequence identity to the sequence of SEQ ID NO: 1. In some embodiments, the isolated ubonodin peptides comprises the amino acid sequence of SEQ ID NO: 1.

In another aspect, the present invention provides pharmaceutical compositions comprising an ubonodin peptide and a pharmaceutically acceptable carrier.

In another aspect, the present invention provides recombinant nucleic acids comprising a nucleotide sequence encoding an ubonodin peptide that comprises an amino acid sequence having at least 70% sequence identity to the sequence of SEQ ID NO: 1.

In a further aspect, the present invention provides methods of treating a Burkholderia infection in a subject in need thereof comprising administering to the subject an ubonodin peptide comprising an amino acid sequence having at least 70% sequence identity to the sequence of SEQ ID NO: 1. In some embodiments, the ubonodin peptide comprises an amino acid sequence having at least 75%, at least 80%, at least 85%, at least 90%, at least 95% or 100% sequence identity to the sequence of SEQ ID NO: 1. In certain embodiments, the ubonodin peptide comprises an amino acid sequence of SEQ ID NO: 1.

In some embodiments, the ubonodin peptide comprises a substitution in the sequence of SEQ ID NO: 1 selected from the group consisting of a G28C substitution, a Y26F substitution, a H15A substitution, a H17A substitution, and combinations thereof.

In some embodiments, the ubonodin peptide is 26 to 30 amino acids in length. In certain embodiments, the ubonodin peptide is 27 to 29 amino acids in length. In certain embodiments, the ubonodin peptide is 28 amino acids in length.

In some embodiments, the Burkholderia infection is a Burkholderia thailandensis infection, Burkholderia multivorans infection, Burkholderia ubonensis infection, Burkholderia ambifaria infection, Burkholderia anthina infection, Burkholderia arboris infection, Burkholderia cenocepacia infection, Burkholderia cepacia infection, Burkholderia contaminans infection, Burkholderia diffusa infection, Burkholderia dolosa infection, Burkholderia lateens infection, Burkholderia lata infection, Burkholderia metallica infection, Burkholderia pyrrocinia infection, Burkholderia seminalis infection, Burkholderia stabilis infection, Burkholderia uronensis infection, Burkholderia vietnamiensis infection, Burkholderia mallei infection, or a combination thereof.

In certain embodiments, the Burkholderia infection is a lung infection.

In some embodiments of the methods disclosed, the subject is a human subject.

In some embodiments, the human subject has cystic fibrosis.

In some embodiments of the methods disclosed, the subject is a non-human animal subject.

In some embodiments of the methods disclosed, the methods further comprise administering to the subject one or more antibiotics selected from the group consisting of amikacin, azithromycin, aztreonam, tobramycin, levofloxacin, vancomycin, molgramostim, nitric oxide, gallium, SPI-1005, ALX-009 and SNSP113.

In some embodiments, the one or more antibiotics are administered to the subject simultaneously with the ubonodin peptide. In some embodiments, the one or more antibiotics are administered to the subject before the administration of the ubonodin peptide. In some embodiments, the one or more antibiotics are administered to the subject after the administration of the ubonodin peptide.

In certain embodiments, the one or more antibiotics and the ubonodin peptide are administered to the subject in the same composition.

In some embodiments, the ubonodin peptide is administered by inhalation, intravenously or orally, or a combination thereof.

In another aspect, the present invention provides recombinant nucleic acids comprising:

a first nucleotide sequence having at least 70% sequence identity to the sequence of SEQ ID NO: 2, wherein the first nucleotide sequence is operably linked to a first promoter;

a second nucleotide sequence having at least 70% sequence identity to the sequence of SEQ ID NO: 3;

a third nucleotide sequence having at least 70% sequence identity to the sequence of SEQ ID NO: 4;

and a fourth nucleotide sequence having at least 70% sequence identity to the sequence of SEQ ID NO: 5,
wherein the second, third and fourth nucleotide sequences are operably linked to a second promoter.

In some embodiments, the first promoter is an inducible promoter such as, e.g., an IPTG-inducible T5 promoter. In certain embodiments, the IPTG-inducible T5 promoter comprises a nucleic acid sequence having at least 70% sequence identity to the sequence of SEQ ID NO: 6.

In some embodiments, the second promoter is a constitutive promoter such as, e.g., a promoter from a microcin J25 gene cluster. In some embodiments, the constitutive promoter comprises a nucleic acid sequence having at least 70% sequence identity to the sequence of SEQ ID NO: 7.

In certain embodiments, the first nucleotide sequence is downstream of the first promoter. In some embodiments, the second, third and fourth nucleotide sequences are downstream of the second promoter.

In some embodiments, the recombinant nucleic acid comprises a bacterial expression vector.

In some embodiments, the recombinant nucleic acid comprises a nucleic acid sequence having at least 70% sequence identity to the sequence of SEQ ID NO: 8. In other embodiments, the recombinant nucleic acid comprises a nucleic acid sequence having at least 75%, at least 80%, at least 85%, at least 90%, at least 95% or 100% sequence identity to the sequence of SEQ ID NO: 8.

In some embodiments, the first nucleotide sequence of the recombinant nucleic acid comprises a nucleic acid sequence having at least 75%, at least 80%, at least 85%, at least 90% or at least 95% sequence identity to the sequence of SEQ ID NO: 2.

In some embodiments, the second nucleotide sequence of the recombinant nucleic acid comprises a nucleic acid sequence having at least 75%, at least 80%, at least 85%, at least 90% or at least 95% sequence identity to the sequence of SEQ ID NO: 3.

In some embodiments, the third nucleotide sequence of the recombinant nucleic acid comprises a nucleic acid sequence having at least 75%, at least 80%, at least 85%, at least 90% or at least 95% sequence identity to the sequence of SEQ ID NO: 4.

In some embodiments, the fourth nucleotide sequence of the recombinant nucleic acid comprises a nucleic acid sequence having at least 75%, at least 80%, at least 85%, at least 90% or at least 95% sequence identity to the sequence of SEQ ID NO: 5.

In another aspect, the present invention provides host cells comprising the recombinant nucleic acids described by the present disclosure.

In some embodiments, the host cell is a bacterial cell such as, e.g., an Escherichia coli cell.

In another aspect, the present disclosure provides methods of making an ubonodin peptide, comprising expressing a recombinant nucleic acid as described in the present disclosure in a host cell; and obtaining the expressed ubonodin peptide from the host cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows the sequence and structure of ubonodin. Top: ubonodin is the largest lasso peptide discovered at 28 aa. Bottom: the NMR structure of ubonodin reveals a remarkable 18 aa loop and a short 2 aa tail. The sidechains of steric lock residues Tyr-26 and Tyr-27 are highlighted. Additional views of the structure are in FIG. 9.

FIG. 2 shows antimicrobial activity of ubonodin. A: Autoradiograph of abortive transcription initiation assays showing that ubonodin inhibits E. coli RNA polymerase. The heading in each gel lane is the concentration of ubonodin added to the assay in μM. CpApU* is the abortive transcript product. B: Spot-on-lawn assay showing the antimicrobial activity of ubonodin against Burkholderia multivorans. Concentration of ubonodin in each spot is given on the figure.

FIG. 3 shows mutagenesis of ubonodin. A: Left: tolerance of ubonodin to amino acid substitutions. While all 13 single amino acid variants could be detected by LC-MS, only 6 were produced at a level sufficient for purification. The production level is coded as follows: green is at or near wild-type levels, yellow is less than 20% of wild-type, and red is only detectable by LC-MS. B: Antimicrobial activity of purified ubonodin variants. The H15A, H17A, and Y26F variants have near wild-type activity (green) while the G28C variant is less potent than wild-type. See also FIGS. 12 and 13 for traces and spot assays on these variants.

FIG. 4 shows lasso peptide biosynthesis and refactoring of ubonodin gene cluster. A: cartoon of lasso peptide biosynthesis. The precursor protein, A, is cleaved and cyclized by the B and C enzymes, respectively. The D protein, an ABC transporter, pumps the mature lasso peptide out of the cell. B: Refactoring of the ubonodin gene cluster. The uboA gene was assembled from short oligonucleotides. The uboBCD operon was codon optimized and assembled from three gBlocks (˜1500 bp each). The uboA gene was cloned under the control of an IPTG-inducible T5 promoter while the uboBCD operon was cloned downstream of a constitutive promoter found in the microcin J25 gene cluster.

FIG. 5 shows MS² analysis of ubonodin. The [M+3H]³⁺ ion of ubonodin (monoisotopic mass of 1066.8022) was subjected to fragmentation by CID. The major peak observed is the parent ion corresponding to intact ubonodin.

FIG. 6 shows 2D NMR spectra of ubonodin. A: gCOSY spectrum, B: TOCSY spectrum, C: NOESY spectrum with 100 ms mixing time used for calculation of distance restraints. D: NOESY spectrum with 500 ms mixing used for peak assignments.

FIG. 7 shows visualizations of the ubonodin NMR structure. A: different rotations of the top ubonodin structure showing the relative compactness of the 18 aa loop. B: Alignment of the top 20 NMR structures showing strong similarity of the structures in the isopeptide-bonded ring and tail regions but less similarity in the loop region. The Tyr-26 and Tyr-27 sidechains are shown in all figures.

FIG. 8 shows NMR structures of other large lasso peptides. Sphingopyxin I (x-ray structure, PDB SJQF) and astexin-3 (NMR structure, PDB 2N6V) are characterized by relatively short loop regions and long tails. Note that full-length sphingopyxin I has five additional amino acids appended to its C-terminal tail. These topologies are in stark contrast to the structure of ubonodin, which has an 18 aa loop and only a 2 aa tail. Refer to FIG. 7 for the ubonodin structure.

FIG. 9 shows a comparison of the NMR structures of RNA polymerase-inhibiting antimicrobial lasso peptides. Two different structures of microcin J25 deposited in the PDB are presented as are the structures of citrocin and ubonodin. All of the structures have high similarity in the ring and tail regions, but great variability in the loop region.

FIG. 10 Top: polyacrylamide gels of three replicates of in vitro abortive transcription assay. Blue bars represent a splice point in the gels. The concentration of ubonodin or microcin J25 used in each assay is presented in the lane headings. CpApU* represents the abortive transcript product while U* is α-³²P UTP. The microcin J25 lanes have been previously published in Figure S9 of reference 14 of Cheung-Lee et al. JBC 2019, 294, 6822 and are presented here for comparison purposes with ubonodin and to show the transcript level with no peptide inhibitor. Bottom: quantification of the gel images. Each of the three data points are shown in circles, the mean is shown in diamonds, and the error bar represents the standard deviation.

FIG. 11 shows a phylogenetic tree of representative strains tested for susceptibility to ubonodin and the natural producer organism of ubonodin, B. ubonensis. Burkholderia cepacia complex (Bcc) members are highlighted in red. Branch lengths are shown proportional to genetic change.

FIG. 12 shows HPLC traces of crude supernatant extracts of ubonodin variants. The identity of each variant is presented on the trace as is the isolated yield when determined. For reference, the yield of wild-type ubonodin was 1.8 mg/L. Note that the peaks for the I6L and I21L variants of ubonodin are broad. Note also that the extracellular metabolome of cells producing the Y26F and G28C variants differs substantially from the other variants.

FIG. 13 shows activity of ubonodin variants. Spots of 2-fold serial dilutions were placed on the plate in a clockwise direction, and the spots are labeled on each plate. Arrows indicate the last spot that caused inhibition of growth. For reference, wild-type ubonodin has a minimal inhibitory concentration of 7.8 μM using this same assay (see FIG. 2).

FIG. 14 shows ubonodin thermostability as assessed via a spot-on-lawn activity assay. Ubonodin was heated at either 50° C. or 95° C. for 0, 2, 4, or 6 hours. Ten microliter samples of the seven heating conditions were used in an antimicrobial activity test against Burkholderia multivorans. N/A: not applicable.

FIG. 15 shows ubonodin thermostability at 95° C. analysis via LC-MS. A) Total ion current (TIC) chromatograms of unheated ubonodin, unheated ubonodin treated with carboxypeptidase, ubonodin heated for 2 hours, and carboxypeptidase digestion of ubonodin after heating for 2 hours. B-C) TIC chromatogram of the heated ubonodin sample with major picks labeled and corresponding table of masses detected. Glu-8 is colored yellow to indicate the presence of an isopeptide bond.

FIG. 16 shows schematic showing cleavage degradation products of heat-treated ubonodin. Intact ubonodin (center), can be cleaved at all the Asp residues (2 in loop, 1 in ring), generating a series of [2]rotaxane and branched peptides. Mass spectrometry evidence was seen for all species except the one boxed in red.

FIG. 17 shows MS/MS spectra of heat-treated ubonodin cleavage products. A-C) Cartoon shows the parent ion species that was fragmented. Assigned daughter ions are annotated on the MS/MS spectrum. Nomenclature for the daughter ions are indicated above the spectrum. Glu-8 is shown in yellow to indicate the isopeptide bond and residues in red is where heat-cleavage occurred.

FIG. 18 shows carboxypeptidase analysis of ubonodin thermal stability. A) TIC chromatograms of carboxypeptidase-treated samples of intact ubonodin and ubonodin heated at 95° C. Major peaks are labeled, with peaks sharing the same retention times and masses sharing a label. B) Corresponding loop cleavage products detected. Note that peaks 1, 2, and 4 are non-C-terminal cleavage products of the intact lasso peptide by promiscuous activity of carboxypeptidase at I16 and Q20. Masses for peaks 5 and 6 are consistent with carboxypeptidase products of the heat-cleaved peptide at Asp-23, while the mass for peak 7 is consistent with a carboxypeptidase product of the heat-cleaved peptide at Asp-18.

### DETAILED DESCRIPTION

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting. As used herein, the indefinite articles “a”, “an” and “the” should be understood to include plural reference unless the context clearly indicates otherwise.

The present disclosure discloses lasso peptides, a class of ribosomally synthesized and post-translationally modified (RiPP)⁸ products defined by their chiral rotaxane structure established via formation of an isopeptide bond between the peptide N-terminus and an acidic sidechain.⁹⁻¹⁰ One lasso peptide, capistruin, was isolated from Burkholderia thailandensis and shown to have antimicrobial activity against phylogenetically related species.¹¹ The present disclosure provides a potent antimicrobial lasso peptide with an unprecedented structure encoded in a Burkholderia genome.

In one aspect, the present invention provides isolated ubonodin peptides comprising an amino acid sequence having at least 70% sequence identity to the sequence of SEQ ID NO: 1 (GGDGSIAEYFNRPMHIHDWQIMDSGYYG). In some embodiments, the peptide comprises an amino acid sequence having at least 70% sequence identity to the sequence of SEQ ID NO: 1, but does not consist of SEQ ID NO:1. In some embodiments, the ubonodin peptide comprises an amino acid sequence having at least 75%, at least 80%, at least 85%, at least 90% or at least 95% sequence identity to the sequence of SEQ ID NO: 1.

As used herein, the term “identity” or “identical” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

In another aspect, the present invention provides pharmaceutical compositions comprising an ubonodin peptide and a pharmaceutically acceptable carrier.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

In another aspect, the present invention provides recombinant nucleic acids comprising a nucleotide sequence encoding an ubonodin peptide that comprises an amino acid sequence having at least 70% sequence identity to the sequence of SEQ ID NO: 1.

In a further aspect, the present invention provides methods of treating a Burkholderia infection in a subject in need thereof comprising administering to the subject an ubonodin peptide comprising an amino acid sequence having at least 70% sequence identity to the sequence of SEQ ID NO: 1. In some embodiments, the ubonodin peptide comprises an amino acid sequence having at least 75%, at least 80%, at least 85%, at least 90%, at least 95% or 100% sequence identity to the sequence of SEQ ID NO: 1. In certain embodiments, the ubonodin peptide comprises an amino acid sequence of SEQ ID NO: 1.

The term “treatment” or “treating” as used within the context of the present invention is meant to include therapeutic treatment as well as prophylactic, or suppressive measures for the infection. Thus, for example, the term treatment includes the administration of an agent prior to or following the onset of an infection thereby preventing or removing all signs of the infection. As another example, administration of the agent after clinical manifestation of the infection to combat the symptoms comprises “treatment” of the infection.

In some embodiments, the ubonodin peptide is administered to a subject who has a Burkholderia infection (e.g., an infection with a Burkholderia cepacia complex (Bcc) strain). In other embodiments, the ubonodin peptide is administered to a subject who is at risk for developing a Burkholderia infection (e.g., at risk for developing an infection with a Bcc strain), such as a subject who has cystic fibrosis.

In some embodiments, the ubonodin peptide comprises a substitution in the sequence of SEQ ID NO: 1 selected from the group consisting of a G28C substitution, a Y26F substitution, a H15A substitution, a H17A substitution, and combinations thereof.

In some embodiments, the ubonodin peptide is 26 to 30 amino acids in length. In certain embodiments, the ubonodin peptide is 27 to 29 amino acids in length. In certain embodiments, the ubonodin peptide is 28 amino acids in length.

In some embodiments, the Burkholderia infection is an infection with a Bcc strain. In some embodiments, the Burkholderia infection is a Burkholderia cepacia infection, Burkholderia thailandensis infection, Burkholderia multivorans infection, Burkholderia ubonensis infection, Burkholderia ambifaria infection, Burkholderia anthina infection, Burkholderia arboris infection, Burkholderia cenocepacia infection, Burkholderia contaminans infection, Burkholderia diffusa infection, Burkholderia dolosa infection, Burkholderia lateens infection, Burkholderia lata infection, Burkholderia metallica infection, Burkholderia pyrrocinia infection, Burkholderia seminalis infection, Burkholderia stabilis infection, Burkholderia uronensis infection, Burkholderia vietnamiensis infection, Burkholderia mallei infection, or a combination thereof.

In particular embodiments, the Burkholderia infection is a Burkholderia cepacia infection.

In certain embodiments, the Burkholderia infection is a Burkholderia multivorans infection.

In some embodiments, the Burkholderia infection is a lung infection.

In some embodiments of the methods disclosed, the subject is a human subject. In some embodiments, the human subject has cystic fibrosis.

In some embodiments of the methods disclosed, the subject is a non-human animal subject.

In some embodiments of the methods disclosed, the methods further comprise administering to the subject one or more antibiotics selected from the group consisting of amikacin, azithromycin, aztreonam, colistin, tobramycin, levofloxacin, vancomycin, molgramostim, nitric oxide, gallium, SPI-1005, ALX-009 and SNSP113.

In some embodiments, the one or more antibiotics are administered to the subject simultaneously with the ubonodin peptide. In some embodiments, the one or more antibiotics are administered to the subject before the administration of the ubonodin peptide. In some embodiments, the one or more antibiotics are administered to the subject after the administration of the ubonodin peptide.

In certain embodiments, the one or more antibiotics and the ubonodin peptide are administered to the subject in the same composition (e.g., an antibiotic cocktail). In certain embodiments, the one or more antibiotics and the ubonodin peptide are administered to the subject in separate compositions.

In some embodiments, the ubonodin peptide is administered by inhalation, intravenously or orally, or a combination thereof. In certain embodiments, the ubonodin peptide is administered by inhalation or injection (e.g., by i.v. injection) as a single active agent (e.g., in the absence of other antibiotics).

In particular embodiments, the ubonodin peptide is included in a formulation (e.g., an antibiotic cocktail, such as a cocktail comprising amikacin, aztreonam, colistin, and tobramycin) with one or more additional active agents (e.g., an antibiotic, such as amikacin, aztreonam, colistin, and tobramycin), wherein the formulation is administered by inhalation.

In another aspect, the present invention provides recombinant nucleic acids comprising:

a first nucleotide sequence having at least 70% sequence identity to the sequence of SEQ ID NO: 2, wherein the first nucleotide sequence is operably linked to a first promoter;

a second nucleotide sequence having at least 70% sequence identity to the sequence of SEQ ID NO: 3;

a third nucleotide sequence having at least 70% sequence identity to the sequence of SEQ ID NO: 4;

and a fourth nucleotide sequence having at least 70% sequence identity to the sequence of SEQ ID NO: 5,
wherein the second, third and fourth nucleotide sequences are operably linked to a second promoter.

As used herein “recombinant nucleic acid” refer to nucleic acids that are obtained by recombinant means, e.g., the cloning of nucleic acid sequences from a recombinant library or a cell genome, using ordinary cloning and amplification technology, and the like, or by synthetic means.

As used herein, a “promoter” is a region of DNA that initiates transcription of a particular gene/nucleic acid sequence.

As used herein, the phrase “operably linked” means that the nucleic acid is positioned in the recombinant nucleotide, e.g., vector, in such a way that enables expression of the nucleic acid under control of the element (e.g., promoter) to which it is linked.

SEQ ID NO: 2
ATGAAAAATCGTAGCACCAAAGAGAGCTTCGAAATTACCTGCATTGGCG
ATGTGGATGTGATTACCCTGATGCAGGATGCGAGCCGTGCGACAATGGG
AGGCGATGGCAGCATTGCGGAATACTTTAACCGTCCGATGCATATTCAT
GATTGGCAGATTATGGATAGCGGCTATTATGGCTGA
SEQ ID NO: 3
ATGCCGTATGCGCTGAGCCAGCATGCGCGTCTGGCGTGTTATGAAGATG
ATCTGATTATTCTGACCATTCGTGATAATCGTTTTCATCTGATCAAAGA
TGTGAGCCGTGATGCGGTGGACGCGTTATATGAACCGATGGCGGGACAG
CGTGGCGCAGGACTGCATGACGCGCTTCGTATTATGGGCGTGCTGGAAG
AGAGTCGCGATCGTGCGGATATTCCGCCTGCGGGACTGCGTCCGAAAAG
CTATGTGGAACAGCGTTGGATGATGCCGCTGACTCGGCATGCTCCGGCG
ACCTTAGTGGGCACCGTGGCGTCGCTGGTGGCACTGTATCGTGCAACCC
TGATGATTAAACTGGGCGGCTTTCGTCGTATTGTGAGCATTGGCAAATG
GCCGGCTCGTATGGCGAGCGGCAGTGTCGATGTGGATGGCACAGTGCAG
GCTGCAATGGGCGATCTGAACCGTGTGTTTGCGTGCGATGTGTCTGGCA
ATCGTTGCCTGACCTATAGCCTGGCGCTGACCCTGCTGCTGCGTCGTAA
AATTCCGAATGTTTCACTGGTGGTGGGCGTCCGTACCCGTCCGTTTTTT
AGCCATGCGTGGGTTGAAGTGGACGGTCGCGTGGTGAATGATACCGCGG
ATCTGCGTAAAAATCTGGCGGTGATCCTGGAGGTTTGA
SEQ ID NO: 4
ATGTTCATTGCCTACCCTGAGAACATAGCGAAGCATTTGGAATACATCA
TTGATGAATGGGCTGGTCGTAATCGTCTGACCACCCACCTGCATGGTAA
GTTCGTGGTGCGTTGTAGCGATCGTTGGACCGTGAGCAAGTGGGGCAAT
TTCATTGAGTTCTTTGAAGGGATGGCTTATGAGTGGCCGACCTGCCAGG
CTTGGCCAGCGCCGGAGCGTCGTGCTCGTCTTAGTAATAGCATTGGCTA
TTTTACCAGCATTCTGTTGGATTCCGATACCCTGGAAGTCGTGCGCAGC
CTGTATCGTGCGACCGACATCTTTTATACCGAAAGCGATGGCATGATGT
TAGCGTGTAGTGAACTGGCTGTGGTGGTCGCGCTTCGTGGCGGATTTGC
ACGCCAGCGTATTGATGTGGATTATTGCCATGATTTCATAGCACACCAA
CAAAAGTTCGACGGACATACGTCATTTGAATCAATTAACGAGGTGATGC
TGGGCGAATGTATACGGATGAGCACAAGCGATATCATTTCAGCTGCGTT
TGTGAATCGTCCGATTGTCCCGAGCGGCGACATCGTGGACACCTTGCGT
GATACCTTAGCGGCATTTACACGTCCATTTGATGGCACCGTCCTGATGT
TTAGCGGGGGTCTGGATAGCAGTACCCTGTTGTGGACTCTGCTGGAATC
TGGCACTAAACCGCTGGTGCTGCACAGCGAGTCTGGGCCGGATGCGCGT
GACAGCGAATACCAGGACGCAGCGGCAGTGGCACTGGATCTGGGCTGCG
AAATTCAGCGTTTCGTGCCGGGACGCGAGGACTATAGCCGCGCTTTTAC
TATCAGTGATGACGGCCAAAGCAGCAGTCCGTATGATATTCCCATCTTC
CTGTCTCGTAGCTCTGCTCGTTCGGGCTTATCTATCGATGAAACCAGCC
TGCTGGTGACCGGGCATGGGGGTGACCATGTGTTCGTGCAGAACCCCGA
AAACAACTCCTGCTTGGCGGCTCTTCAAGCGGGACGGGTGTTTGAGTAT
CTGCGTACGGTGCGTAAACTGAGCCGTCTGAAAGGCCGTCGGGGCGTGG
AGATTGTACGGCATAACCTGCGCCTGCTTATGGGAGGCCATCTGCTGTC
AGGTTCGTTCCCGGATTGGCTGCCGCGTCCGCGGCACCGCTCCGCACGT
CGGACAGGCCACTACTTAATTCGCGACCTGGACCGGCGTATGGCGAAAC
ATACTCATCTGAGCGCCATTCTGCAGGCTTTACAGAGCGCAAGCATTCC
GCGCAACGGACCGCCCATGTTGGCGCCGTTGTTGCTGCAAAATGTGATT
GGCCATATGATGGGCATACCGGTTCAGGATACGTTTACTGAAACCCATG
ATCGTGTGACACTTCGTGAGTCGATTTATCGTCAGTCTGGCAAATCTTT
TGCGTGGCGTCGTACCAAACGTGCGTCCAGCGCTTTCCTGTTTGAACTG
TTGAGCCAGTCCGAAGTGAATTTAGCCGATCTGATCGACCGCAGCCACT
TTGTTCCACTGTTGCATATTGATCGTCGGGCATTACTGGCGGAAGTGCG
TCAGAATTGCCGGATAGCGCTGACCGGCAACTTTAAACATATTGTCAAC
TTGTATAAGATTGAAGCGCACCTTCGCTCAATAGAGCATCAGTCTGCAG
AACTAACCAGACCATGA
SEQ ID NO: 5
ATGAAGCGTTGGATCGGTATCTATTCTGAGATCGGCCACCATTTGCAAC
GCCAGGAACGGTACTTTGTAGTAGCAATTCTGTTTTGCACCCTTGGTGC
TGCGGCCAGCATGGCGATGAGCCCGGTGTTTTTAGGGCGTCTGGCGGAT
TCACTGCTTGCGGCGGATCGTCGTATGCCCGCGTACATTATCTACTTAG
CGGCAAGCTATTTGATCACCATTGCTATGCCAAAGCTGCTGGGCACCGT
AGATCTGTACCTGCAGTCAATGTTGCGTTTACGTGCGAACCGTAGCCTG
TTAGCCGGGTACTTCAACTATCTGTGTCGGCAACCCGAGAGTTTTTTCG
TGAATAAGAATAGTGGTGAGCTTACCCAAGAGATCACCCAAGCGTCTAA
TGATCTTTACCTGATTGTACGGAACCTGACCACTAGCCTTATCTCGCCG
ATTGTGCAGGTGAGCATTGCGGTGGTCGTCCTTGCGAGCAATCATGACC
TGTTGGTGGCGGGGACGATAGCGATTTATGTGGCTTTGTTCGTAACAAA
CAATGTAATACATGGCCGTCGTTTGGTAGAACTGAAATTCCGTTGCATG
GATGCAGGTCGGAAAAGCTATGGAACGTTGACGGACAGCATCACCAATA
TTCAGGTGGCGCGTCAGTTTAATGGGTATCGTTTCCTGTTGAGCCGCTA
TCAACGGGTGCTTGACGAAGACCGTCATACACAGGGCGACTACTGGAAG
ATCTCTCTGCGTATGCAGTTTTTCAACGCGTGTCTGTTTGTGGGCCTGT
TTGGCGTAACCTTTCTGATGGCGCTGCACGAAGTAGTGACCGGTGCGCG
CTCTATTGGCAATTTCGTGCTTGTCGCCGCGTATACCGTGACCCTGTTA
AGCCCCATCGAGATTCTGGGCAATATGTTTACCGAAATTAACCAGAGTC
TGGTGACCTTTGGGCGTTTTCTTGATAAATTGTCAGCAGCCACAGCTCC
TCTTAGCCAGCGTGCGCCTAAGCCGGCAGTTAAGTCCGCGGCACCGGCT
ATCGAATTTGAACGTGTGTGCGTGACCTATCCGGGTGCCAATCGCCAGG
CATTAACTGATGTGGGCTTTACAGTGGATGCCGGAAAGCGTGTAGCGAT
AACTGGTCCCTCTGGAGCAGGCAAGAGCAGCCTGGTGAAAGTTCTGACC
CGCCAACTTGTGGCGGAAGAAGGAGCCATTCGTATTTTTGGCGAGGATA
TCTTATGTATTGATGCGCAGACCCTGAGCGAACGTATTGGCTGCGTGTC
ACAGGACGTACTGCTGTTTAAAGATACCTTACGGTTTAACTTGCAGATT
TCGCGCCCTGATGCTTCGGACGCGGACATGGTCACTGCACTTGAGTGCG
CGGGACTGACGGATCTTTTAGTGGACTTACCTGCCGGGTTGGACACGAT
GTTAGGCGATCGTGGCGCAACACTTTCTGGAGGTCAGCGTCAGCGGTTG
GCGTTAGCCCGCTTGTTCCTGCGTGCCCCCGACATTGTGTTGGTTGATG
AAGGCACCTCTTCGCTGGATTTGGTAACTGAGCAGTATGTTCTGGACAA
GGTGTTTGAAGTGTTTAGCGACAAAACCATAGTGATGATAACCCACCGT
CCTAGCGCGATGACCAAAGTTGATGCCGTGATTATCATGAGCGATGGTC
GTATTGACGATCATGCGGAACCGGATGTGCTTCGTAGCCGTAATACCTT
TTTTGCGCGTGTTGTGGAATCTTCTTTGCGTTGA

In some embodiments, a first nucleotide sequence comprises the nucleotide sequence of uboA (SEQ ID NO: 2). In some embodiments, a second nucleotide sequence comprises the nucleotide sequence of uboB (SEQ ID NO: 3). In some embodiments, a third nucleotide sequence comprises the nucleotide sequence of uboC (SEQ ID NO: 4). In some embodiments, a fourth nucleotide sequence comprises the nucleotide sequence of uboD (SEQ ID NO: 5).

In some embodiments, the first promoter is an inducible promoter such as, e.g., an IPTG-inducible T5 promoter. In certain embodiments, the IPTG-inducible T5 promoter comprises a nucleic acid sequence having at least 70% sequence identity to the sequence of SEQ ID NO: 6 (TCATAAAAAATTTATTTGCTTTGTGAGCGGATAACAATTATAATA). In some embodiments, the inducible promoter is from the pQE-80 vector.

In some embodiments, the second promoter is a constitutive promoter such as, e.g., a promoter from a microcin J25 gene cluster. In some embodiments, the constitutive promoter comprises a nucleic acid sequence having at least 70% sequence identity to the sequence of SEQ ID NO: 7 (CATCAATTAAGAAAAAAATTTAGCTTGTAGATAAATTCAGAAGTTTTATTATTCC AATTGAGTGTAAAGGCATAACTACAGGAGGGAGTGTGCAAA).

In certain embodiments, the first nucleotide sequence is downstream of the first promoter. In some embodiments, the second, third and fourth nucleotide sequences are downstream of the second promoter.

In some embodiments, the recombinant nucleic acid comprises a bacterial expression vector.

In some embodiments, the recombinant nucleic acid comprises a nucleic acid sequence having at least 70% sequence identity to the sequence of SEQ ID NO: 8. In other embodiments, the recombinant nucleic acid comprises a nucleic acid sequence having at least 75%, at least 80%, at least 85%, at least 90%, at least 95% or 100% sequence identity to the sequence of SEQ ID NO: 8.

SEQ ID NO: 8
TCATAAAAAATTTATTTGCTTTGTGAGCGGATAACAATTATAATAGATT
CAATTGTGAGCGGATAACAATTTCACACAGAATTCATTAAAGAGGAGAA
ATTAACTATGAAAAATCGTAGCACCAAAGAGAGCTTCGAAATTACCTGC
ATTGGCGATGTGGATGTGATTACCCTGATGCAGGATGCGAGCCGTGCGA
CAATGGGAGGCGATGGCAGCATTGCGGAATACTTTAACCGTCCGATGCA
TATTCATGATTGGCAGATTATGGATAGCGGCTATTATGGCTGAAAGCTT
AATTAGCTGAGCTTGGACTCCTGTTGATAGATCCAGTAATGACCTCAGA
ACTCCATCTGGATTTGTTCAGAACGCTCGGTTGCCGCCGGGCGTTTTTT
ATTGGTGAGAATCCAAGCTAGCCATCAATTAAGAAAAAAATTTAGCTTG
TAGATAAATTCAGAAGTTTTATTATTCCAATTGAGTGTAAAGGCATAAC
TACAGGAGGGAGTGTGCAAAATGCCGTATGCGCTGAGCCAGCATGCGCG
TCTGGCGTGTTATGAAGATGATCTGATTATTCTGACCATTCGTGATAAT
CGTTTTCATCTGATCAAAGATGTGAGCCGTGATGCGGTGGACGCGTTAT
ATGAACCGATGGCGGGACAGCGTGGCGCAGGACTGCATGACGCGCTTCG
TATTATGGGCGTGCTGGAAGAGAGTCGCGATCGTGCGGATATTCCGCCT
GCGGGACTGCGTCCGAAAAGCTATGTGGAACAGCGTTGGATGATGCCGC
TGACTCGGCATGCTCCGGCGACCTTAGTGGGCACCGTGGCGTCGCTGGT
GGCACTGTATCGTGCAACCCTGATGATTAAACTGGGCGGCTTTCGTCGT
ATTGTGAGCATTGGCAAATGGCCGGCTCGTATGGCGAGCGGCAGTGTCG
ATGTGGATGGCACAGTGCAGGCTGCAATGGGCGATCTGAACCGTGTGTT
TGCGTGCGATGTGTCTGGCAATCGTTGCCTGACCTATAGCCTGGCGCTG
ACCCTGCTGCTGCGTCGTAAAATTCCGAATGTTTCACTGGTGGTGGGCG
TCCGTACCCGTCCGTTTTTTAGCCATGCGTGGGTTGAAGTGGACGGTCG
CGTGGTGAATGATACCGCGGATCTGCGTAAAAATCTGGCGGTGATCCTG
GAGGTTTGATGTTCATTGCCTACCCTGAGAACATAGCGAAGCATTTGGA
ATACATCATTGATGAATGGGCTGGTCGTAATCGTCTGACCACCCACCTG
CATGGTAAGTTCGTGGTGCGTTGTAGCGATCGTTGGACCGTGAGCAAGT
GGGGCAATTTCATTGAGTTCTTTGAAGGGATGGCTTATGAGTGGCCGAC
CTGCCAGGCTTGGCCAGCGCCGGAGCGTCGTGCTCGTCTTAGTAATAGC
ATTGGCTATTTTACCAGCATTCTGTTGGATTCCGATACCCTGGAAGTCG
TGCGCAGCCTGTATCGTGCGACCGACATCTTTTATACCGAAAGCGATGG
CATGATGTTAGCGTGTAGTGAACTGGCTGTGGTGGTCGCGCTTCGTGGC
GGATTTGCACGCCAGCGTATTGATGTGGATTATTGCCATGATTTCATAG
CACACCAACAAAAGTTCGACGGACATACGTCATTTGAATCAATTAACGA
GGTGATGCTGGGCGAATGTATACGGATGAGCACAAGCGATATCATTTCA
GCTGCGTTTGTGAATCGTCCGATTGTCCCGAGCGGCGACATCGTGGACA
CCTTGCGTGATACCTTAGCGGCATTTACACGTCCATTTGATGGCACCGT
CCTGATGTTTAGCGGGGGTCTGGATAGCAGTACCCTGTTGTGGACTCTG
CTGGAATCTGGCACTAAACCGCTGGTGCTGCACAGCGAGTCTGGGCCGG
ATGCGCGTGACAGCGAATACCAGGACGCAGCGGCAGTGGCACTGGATCT
GGGCTGCGAAATTCAGCGTTTCGTGCCGGGACGCGAGGACTATAGCCGC
GCTTTTACTATCAGTGATGACGGCCAAAGCAGCAGTCCGTATGATATTC
CCATCTTCCTGTCTCGTAGCTCTGCTCGTTCGGGCTTATCTATCGATGA
AACCAGCCTGCTGGTGACCGGGCATGGGGGTGACCATGTGTTCGTGCAG
AACCCCGAAAACAACTCCTGCTTGGCGGCTCTTCAAGCGGGACGGGTGT
TTGAGTATCTGCGTACGGTGCGTAAACTGAGCCGTCTGAAAGGCCGTCG
GGGCGTGGAGATTGTACGGCATAACCTGCGCCTGCTTATGGGAGGCCAT
CTGCTGTCAGGTTCGTTCCCGGATTGGCTGCCGCGTCCGCGGCACCGCT
CCGCACGTCGGACAGGCCACTACTTAATTCGCGACCTGGACCGGCGTAT
GGCGAAACATACTCATCTGAGCGCCATTCTGCAGGCTTTACAGAGCGCA
AGCATTCCGCGCAACGGACCGCCCATGTTGGCGCCGTTGTTGCTGCAAA
ATGTGATTGGCCATATGATGGGCATACCGGTTCAGGATACGTTTACTGA
AACCCATGATCGTGTGACACTTCGTGAGTCGATTTATCGTCAGTCTGGC
AAATCTTTTGCGTGGCGTCGTACCAAACGTGCGTCCAGCGCTTTCCTGT
TTGAACTGTTGAGCCAGTCCGAAGTGAATTTAGCCGATCTGATCGACCG
CAGCCACTTTGTTCCACTGTTGCATATTGATCGTCGGGCATTACTGGCG
GAAGTGCGTCAGAATTGCCGGATAGCGCTGACCGGCAACTTTAAACATA
TTGTCAACTTGTATAAGATTGAAGCGCACCTTCGCTCAATAGAGCATCA
GTCTGCAGAACTAACCAGACCATGAAGCGTTGGATCGGTATCTATTCTG
AGATCGGCCACCATTTGCAACGCCAGGAACGGTACTTTGTAGTAGCAAT
TCTGTTTTGCACCCTTGGTGCTGCGGCCAGCATGGCGATGAGCCCGGTG
TTTTTAGGGCGTCTGGCGGATTCACTGCTTGCGGCGGATCGTCGTATGC
CCGCGTACATTATCTACTTAGCGGCAAGCTATTTGATCACCATTGCTAT
GCCAAAGCTGCTGGGCACCGTAGATCTGTACCTGCAGTCAATGTTGCGT
TTACGTGCGAACCGTAGCCTGTTAGCCGGGTACTTCAACTATCTGTGTC
GGCAACCCGAGAGTTTTTTCGTGAATAAGAATAGTGGTGAGCTTACCCA
AGAGATCACCCAAGCGTCTAATGATCTTTACCTGATTGTACGGAACCTG
ACCACTAGCCTTATCTCGCCGATTGTGCAGGTGAGCATTGCGGTGGTCG
TCCTTGCGAGCAATCATGACCTGTTGGTGGCGGGGACGATAGCGATTTA
TGTGGCTTTGTTCGTAACAAACAATGTAATACATGGCCGTCGTTTGGTA
GAACTGAAATTCCGTTGCATGGATGCAGGTCGGAAAAGCTATGGAACGT
TGACGGACAGCATCACCAATATTCAGGTGGCGCGTCAGTTTAATGGGTA
TCGTTTCCTGTTGAGCCGCTATCAACGGGTGCTTGACGAAGACCGTCAT
ACACAGGGCGACTACTGGAAGATCTCTCTGCGTATGCAGTTTTTCAACG
CGTGTCTGTTTGTGGGCCTGTTTGGCGTAACCTTTCTGATGGCGCTGCA
CGAAGTAGTGACCGGTGCGCGCTCTATTGGCAATTTCGTGCTTGTCGCC
GCGTATACCGTGACCCTGTTAAGCCCCATCGAGATTCTGGGCAATATGT
TTACCGAAATTAACCAGAGTCTGGTGACCTTTGGGCGTTTTCTTGATAA
ATTGTCAGCAGCCACAGCTCCTCTTAGCCAGCGTGCGCCTAAGCCGGCA
GTTAAGTCCGCGGCACCGGCTATCGAATTTGAACGTGTGTGCGTGACCT
ATCCGGGTGCCAATCGCCAGGCATTAACTGATGTGGGCTTTACAGTGGA
TGCCGGAAAGCGTGTAGCGATAACTGGTCCCTCTGGAGCAGGCAAGAGC
AGCCTGGTGAAAGTTCTGACCCGCCAACTTGTGGCGGAAGAAGGAGCCA
TTCGTATTTTTGGCGAGGATATCTTATGTATTGATGCGCAGACCCTGAG
CGAACGTATTGGCTGCGTGTCACAGGACGTACTGCTGTTTAAAGATACC
TTACGGTTTAACTTGCAGATTTCGCGCCCTGATGCTTCGGACGCGGACA
TGGTCACTGCACTTGAGTGCGCGGGACTGACGGATCTTTTAGTGGACTT
ACCTGCCGGGTTGGACACGATGTTAGGCGATCGTGGCGCAACACTTTCT
GGAGGTCAGCGTCAGCGGTTGGCGTTAGCCCGCTTGTTCCTGCGTGCCC
CCGACATTGTGTTGGTTGATGAAGGCACCTCTTCGCTGGATTTGGTAAC
TGAGCAGTATGTTCTGGACAAGGTGTTTGAAGTGTTTAGCGACAAAACC
ATAGTGATGATAACCCACCGTCCTAGCGCGATGACCAAAGTTGATGCCG
TGATTATCATGAGCGATGGTCGTATTGACGATCATGCGGAACCGGATGT
GCTTCGTAGCCGTAATACCTTTTTTGCGCGTGTTGTGGAATCTTCTTTG
CGTTGACCATGG

In some embodiments, the first nucleotide sequence of the recombinant nucleic acid comprises a nucleic acid sequence having at least 75%, at least 80%, at least 85%, at least 90% or at least 95% sequence identity to the sequence of SEQ ID NO: 2.

In some embodiments, the second nucleotide sequence of the recombinant nucleic acid comprises a nucleic acid sequence having at least 75%, at least 80%, at least 85%, at least 90% or at least 95% sequence identity to the sequence of SEQ ID NO: 3.

In some embodiments, the third nucleotide sequence of the recombinant nucleic acid comprises a nucleic acid sequence having at least 75%, at least 80%, at least 85%, at least 90% or at least 95% sequence identity to the sequence of SEQ ID NO: 4.

In some embodiments, the fourth nucleotide sequence of the recombinant nucleic acid comprises a nucleic acid sequence having at least 75%, at least 80%, at least 85%, at least 90% or at least 95% sequence identity to the sequence of SEQ ID NO: 5.

In another aspect, the present invention provides host cells (e.g., bacterial cells, mammalian cells, plant cells, or insect cells) comprising the recombinant nucleic acids described by the present disclosure.

In some embodiments, the host cell is a bacterial cell such as, e.g., an Escherichia coli cell.

In another aspect, the present disclosure provides methods of making an ubonodin peptide, comprising expressing a recombinant nucleic acid as described in the present disclosure in a host cell; and obtaining the expressed ubonodin peptide from the host cell.

In another aspect, the present disclosure provides methods of making an ubonodin peptide, comprising expressing a recombinant nucleic acid as described in the present disclosure in an in vitro system; and obtaining the expressed ubonodin peptide. The in vitro system can be any type of system, reagents and/or kits that is utilized to express recombinant nucleic acids in an in vitro environment.

### EXAMPLES

Materials

All restriction enzymes and Q5 polymerase were purchased from New England Biolabs (NEB). Primers and gBlocks were purchased from Integrated DNA Technologies (IDT). The sequences of all primers and gBlocks used in this study are provided in Tables S5 and S6. Solid phase extraction was done using Strata C8 columns from Phenomenex (8B-5005-JCH). All solvents were purchased from Sigma Aldrich. Reverse-phase HPLC was performed using an Agilent 1200 series instrument with a Zorbax 300SB-C18 (9.4 mm ID×250 mm length, 5 μm particle size) column. Mass spectrometry experiments were done using an Agilent 6530 QTOF LC-MS with a Zorbax 300SB-C18 (2.1 mm ID×50 mm length, 3.5 μm particle size) column. Samples were lyophilized using a Labconco FreeZone Freeze Dry System.

Genome Mining

Genome mining was performed using an updated version of our precursor-centric algorithm.¹² The pattern for the precursor was updated to X₁₀₋₄₃TXXX₅₋₇[D/E]X₅₋₂₅ where X is any amino acid. The gene cluster for ubonodin was identified in Burkholderia ubonensis strain MSMB2207. It has subsequently been identified in other Burkholderia ubonensis strains in the NCBI database.

Plasmid Construction

The ubonodin ABCD gene cluster was codon-optimized for E. coli using DNAWorks and refactored for cloning into pQE-80, with the A gene under the T5 promoter and the BCD genes placed under the constitutively-expressed mcjBCD promoter from the microcin J25 gene cluster. First, the uboA gene with an upstream RBS was assembled from six oligonucleotides designed with DNAWorks.³¹ Assembly PCR was done in two steps. An initial PCR with 100 nM of each oligonucleotide was performed to assemble the gene. One microliter of the unpurified product was then used as template for a second round of PCR with 500 nM of the end primers to amplify the assembled product. This gene was then cloned into pQE-80 using EcoRI and HindIII restriction enzymes to generate pWC97.

Three overlapping gBlocks for codon-optimized uboBCD with an upstream mcjBCD promoter and flanking NheI and NcoI restriction sites were designed and purchased. The gBlocks were sequentially assembled with two rounds of overlap PCR where gBlocks 1 and 2 were first overlapped together, purified, and then overlapped with gBlock 3. The assembled product was then cloned into pWC97 using NheI and NcoI restriction enzymes to generate pWC99.

All ubonodin variants were generated using site-directed mutagenesis. Primers used to generate the mutations are provided in Table S6. The uboA variants were then cloned into pWC99 using EcoRI and HindIII restriction enzymes.

Expression and Purification of Ubonodin

The plasmid pWC99 (P_{T5}-uboA P_{mcjBCD}-uboBCD pQE-80) was transformed into Escherichia coli (E. coli) BL21. Typically, a 30 mL LB culture with 100 μg/mL ampicillin was grown overnight. The bacterial density was measured at OD₆₀₀ and used to calculate an OD₆₀₀ measurement of 0.02 for the subculture. The cells were subcultured into 4 L of M9 minimal media with 100 μg/mL ampicillin and supplemented with 40 mg/L of each of the 20 amino acids (8×500 mL cultures in 2 L flasks). The cultures were grown at 37° C., 250 rpm until they reached an OD₆₀₀ absorbance of 0.2. They were then induced with 1 mM IPTG and grown at 20° C., 250 rpm for 20 hours.

The supernatant was then harvested by centrifugation at 4000×g, 4° C., for 20 min and extracted through 6 mL Strata C8 columns. First, each column was activated with 6 mL of methanol and then washed with 12 mL of water. Then 500 mL of supernatant was pumped through the column, which was then washed with 12 mL water and eluted with 6 mL of methanol. The methanol elutions were pooled together and rotavapped dry. The dried extract was then resuspended in 4 mL of 25% acetonitrile/water.

Ubonodin was then purified from the concentrated extract using RP-HPLC. Typically, 60 μL of the extract was injected onto a C18 semi-prep column at a time. An acetonitrile/water gradient with 0.1% trifluoroacetic acid, flowing at 4 mL/min, was used to separate the peptide from other compounds. The gradient used was as follows: 10% acetonitrile from 0 to 1 min, a linear gradient from 10 to 50% acetonitrile from 1 min to 20 min, and a linear gradient from 50 to 90% acetonitrile from 20 min to 25 min. Various fractions were collected. A peak with a retention time of 14.8 min was confirmed using LC-MS to be ubonodin. Purified ubonodin was frozen at −80° C., lyophilized, and then resuspended in water. Concentration was determined using the absorbance at 280 nm with a Nanodrop spectrophotometer using an extinction coefficient of 9530 cm⁻¹ M⁻¹, which was calculated from the amino acid sequence.³²

Ubonodin variants were also expressed and purified similar to the wildtype peptide except on a smaller scale. Typically, 500 mL cultures were grown for each variant. Concentrated extracts and fractions collected from the HPLC for each variant were injected onto the LC-MS to detect production. For variants that produced reasonably well, as judged by the HPLC peak area (ubonodin H15A, H17A, Y26F, and G28C), HPLC purification was performed.

NMR

NMR experiments were done in two different sets. For the first set of experiments, ubonodin was prepared at 9 mg/mL in 95:5 H₂O:D₂O. ¹H-¹H gCOSY, TOCSY and NOESY experiments were conducted at 22° C. using a Bruker Avance III HD 800 MHz spectrometer. TOCSY and NOESY spectra were acquired with 80 and 500 ms mixing time respectively. For the second set of experiments, ubonodin was prepared at 4.6 mg/mL in 95:5 H₂O:D₂O. TOCSY was reacquired with 80 msec mixing time and NOESY were acquired with 100 ms and 40 ms mixing times on the same instrument. Spectra were processed and analyzed using Mnova (Mestrelab). The TOCSY spectra from the two different experiments overlaid well. All residues in the peptide were fully assigned (Table S2). Cross peaks were manually picked and integrated from the 100 msec NOESY spectrum. These cross peak volumes were used as distance constraints in structural calculations done using CYANA 2.1. Seven cycles of combined automated NOESY assignment and structural calculations with 100 initial structures were done, followed by a final structure calculation. The 20 structures with the lowest final target values were then energy-minimized in explicit solvent using GROMACS, using a procedure described by Spronk et al.³³ Each of the 20 structures was placed in a simulation box and solvated with tip3p water. The system was simulated for 4 ps, cooling from 300 K to 50 K.

RNA Polymerase (RNAP) Inhibition Assay

RNAP inhibition was tested using an in vitro abortive initiation assay, as previously described.⁵ Ubonodin was tested in parallel with citrocin and microcin J25.¹⁴ Each 10 μL reaction was set up in triplicate in transcription buffer (100 mM KCl, 10 mM MgCl2, 10 mM DTT, 50 μg/ml BSA, 50 mM Tris, pH 8.0) and contained 125 nM core RNAP, 625 nM σ⁷⁰, 50 nM T7A1 promoter DNA fragment, 500 μM CpA, 100 μM UTP, 0.1 μCi of [α-³²P]UTP, and different concentrations of peptide inhibitor. All incubation and reaction steps were performed at 37° C. First, core RNAP was incubated with σ⁷⁰ for 10 minutes. Then T7A1 promoter DNA was added and incubated for 10 minutes. Next heparin was added to a final concentration of 25 μg/mL along with 0, 1, 10, or 100 μM of peptide and incubated for 10 minutes. RNA synthesis was then initiated with the addition of an NTP mix of 500 μM CpA, 100 μM UTP, and 0.1 μCi of [α-³²P]UTP. After 10 minutes, the reactions were stopped with 2× stop buffer (8 M urea, 1× Tris-borate-EDTA) and heated at 95° C. for 10 minutes. Samples were analyzed on a 23% polyacrylamide gel (19:1 acrylamide:bis-acrylamide). Abortive products were visualized by exposing the gel on a GE storage phosphor screen overnight and digitized using a Typhoon phosphorimaging device. Quantification was done using ImageJ.

Antimicrobial Activity Assay

Antimicrobial activity was tested in two different lab settings. Initial screenings were done against a variety of bacteria with biosafety level (BSL) 2 or below. These screenings were done using a spot-on-lawn inhibition assay, as previously described.³⁴ Briefly, 10 mL of M63 soft agar containing approximately 10⁸ CFUs was overlaid on top of a 10 mL M63 agar plate. After the soft agar solidified, 10 μL spots of twofold peptide dilutions in sterile water were spotted and allowed to dry. The plates were then incubated overnight (30° C. for Burkholderia strains, 37° C. for all other strains tested). All strains tested using this spot-on-lawn inhibition assay with M63 agar is provided in Table S4. The assays involving B. pseudomallei Bp82 were done in the lab of Apichai Tuanyok (Emerging Pathogens Institute, University of Florida). All ubonodin variants were similarly tested using the same method.

Additional testing of ubonodin in liquid and plate assays against Burkholderia strains, including BSL-3 strains, were done at Rutgers Medical School. All of these bacterial strains (see Table S7) were purchased from the American Type Cell Culture Institute (ATCC, Manassas, Va.) or from the Biodefense and Emerging Infections Research Resources Repository (BEI Resources, Manassas, Va.). Bacteria were grown in BBL™ Mueller Hinton II cation adjusted broth (Becton, Dickinson and Company) or agar and incubated overnight in a shaker at 37° C. For the liquid inhibition assay, lyophilized peptide was re-suspended in distilled sterile water and a series of twofold dilutions were prepared and added to a 96 well round bottom cell culture plate (Corning Incorporated, Costar). The highest peptide concentration tested was 100 μg/ml. Stocks of bacterial suspension were prepared by making a 1:1,000 dilution of the overnight bacterial cultures. These bacterial stocks were used to inoculate the 96 well plates to a final volume of 50 μl per well. The plates were incubated for 24 hours at 37° C. The MIC values were determined by observing the presence of pellet in the wells of the plates. The assays were performed in triplicates and the experiments repeated two or three times.

In the plate assays, overnight bacterial cultures were diluted at 1:100, then spread over Mueller-Hinton (MH) agar plates and then 10 μl of peptide solutions at different concentrations were spotted on the MH agar plate starting from a concentration of 500 μg/ml. The plates were incubated for 24 hours at 37° C. and then zones of inhibition were measured.

Thermostability Assay

Ubonodin at 62.5 μM concentration in sterile water was heated at 50 or 95° C. for 0, 2, 4 or 6 hours in a thermocycler. Ten microliter samples at the different temperature and time points were used to perform a spot-on-lawn inhibition assay against Burkholderia multivorans (see antimicrobial activity assay section). Two microliter samples were injected onto LC-MS for stability analysis. LC-MS/MS was also done to identify some of the degradation products.

Additionally, the 0 hour and 2 hour timepoints at 95° C. were digested with carboxypeptidase B and Y in 50 mM sodium acetate, pH 6 for 3 hours (1 unit of each in a 100 μL digestion with ubonodin at 53 μM). Two microliters of the digest was analyzed by LC-MS.

Phylogenetic Tree

16S rRNA sequences were obtained from the NCBI database. The sequences were first aligned using ClustalW. Bayesian phylogenetic analysis was then performed using MrBayes (version 3.2.7a) with the GTR substitution model and gamma-distributed rate variation.³⁵ One million generations were run, sampling every 100th generation. Phylogenetic tree was visualized with Mesquite version 3.6.³⁶

### Results

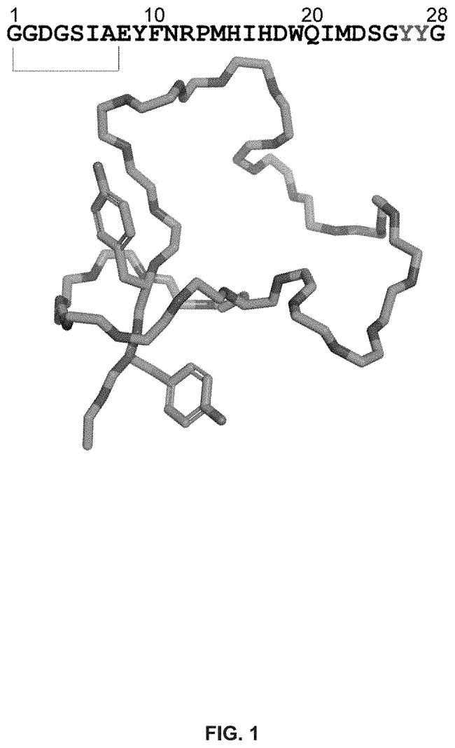

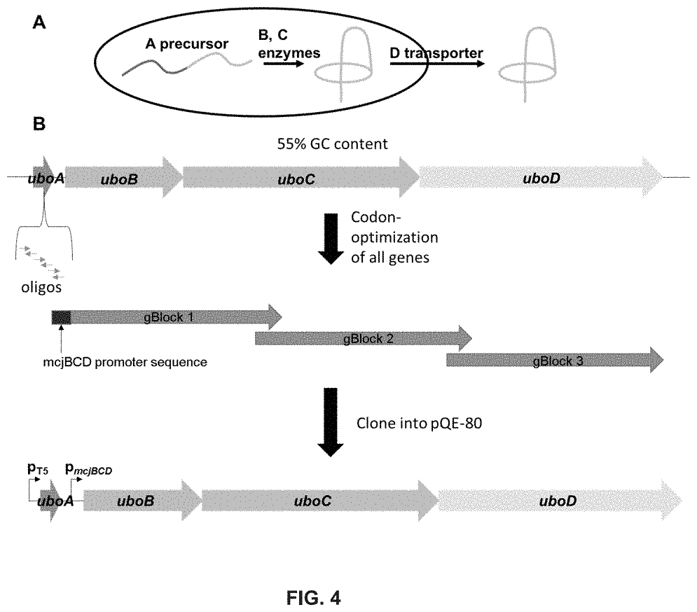

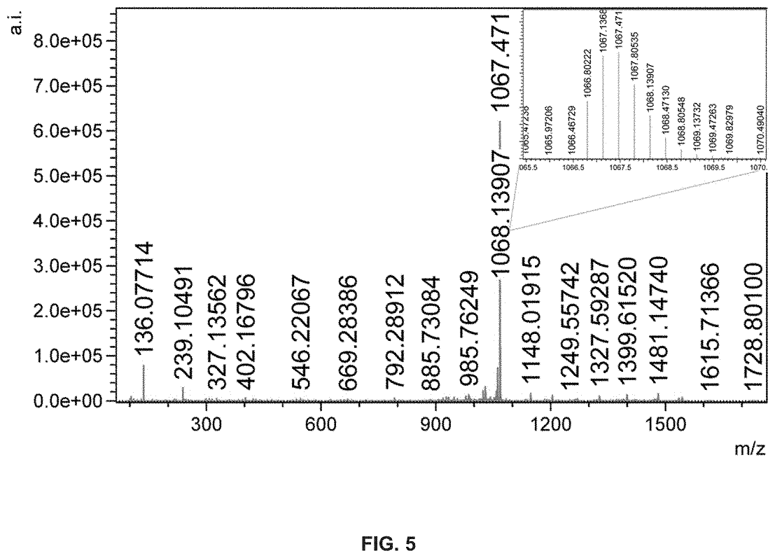

A lasso peptide gene cluster was identified in the organism Burkholderia ubonensis MSMB2207 using a methodology for lasso peptide genome mining.¹²⁻¹³ This cluster also appeared in BLAST searches of the biosynthetic enzymes for citrocin, an antimicrobial lasso peptide produced by strains of Citrobacter.¹⁴ The large size, 28 aa, of the core peptide of this putative lasso peptide (FIG. 1) is longer than any previously characterized example.¹⁰ The lasso peptide gene cluster has 55% GC content, somewhat lower than the GC content of B. ubonensis genomes, which is ˜67%. Currently, there are 306 B. ubonensis genomes in the RefSeq database, and 16 of them harbor this lasso peptide gene cluster (Table 51). The gene cluster was refactored for heterologous expression in E. coli, a strategy that worked well for the production of citrocin.¹⁴ Briefly, the uboA gene encoding the lasso peptide precursor was placed under the control of a strong IPTG-inducible promoter while the uboBCD cassette containing the maturation enzymes and transporter were placed under a constitutive promoter (FIG. 4). The refactored uboABCD gene cluster was introduced into E. coli BL21 which was able to produce 1.8 mg/L of a peptide with a monoisotopic mass of 3197.382 g/mol, which matches well to the predicted mass of the core peptide with one dehydration (3197.376 g/mol). In MS² experiments, this peptide fragmented minimally, similar to what was observed with the lasso peptide microcin J25 (MccJ25)¹⁵⁻¹⁶ (FIG. 5). The peptide, is named ubonodin after the organism that encodes it, B. ubonensis, and the Latin root for knot, nodum.

TABLE S1
Burkholderia ubonensis genomes harboring the ubonodin gene cluster. Start and
stop refer to start and stop codon positions of the ubonodin precursor gene.

| Nucleotide Accession | Start | Stop | Strand | Organism | Strain |
|---|---|---|---|---|---|
| NZ_LOVJ01000074.1 | 69620 | 69802 | + | Burkholderia ubonensis | MSMB1193 |
| NZ_LPCD01000022.1 | 70877 | 71059 | + | Burkholderia ubonensis | MSMB2014WGS |
| NZ_LOZC01000017.1 | 9438 | 9620 | − | Burkholderia ubonensis | MSMB2054 |
| NZ_LOZD01000055.1 | 8632 | 8814 | − | Burkholderia ubonensis | MSMB2055 |
| NZ_LOZH01000036.1 | 9064 | 9246 | − | Burkholderia ubonensis | MSMB2061 |
| NZ_LOZJ01000020.1 | 15910 | 16092 | − | Burkholderia ubonensis | MSMB1754 |
| NZ_LPAE01000160.1 | 56155 | 56337 | + | Burkholderia ubonensis | MSMB1586WGS |
| NZ_LPAI01000131.1 | 8736 | 8918 | − | Burkholderia ubonensis | MSMB1598WGS |
| NZ_LPDR01000062.1 | 32669 | 32851 | + | Burkholderia ubonensis | MSMB1145WGS |
| NZ_LPDV01000100.1 | 56147 | 56329 | + | Burkholderia ubonensis | MSMB1173WGS |
| NZ_LPEN01000085.1 | 8706 | 8888 | − | Burkholderia ubonensis | MSMB1264WGS |
| NZ_LPFT01000052.1 | 35929 | 36111 | + | Burkholderia ubonensis | MSMB1508WGS |
| NZ_LPGA01000029.1 | 56026 | 56208 | + | Burkholderia ubonensis | MSMB1518WGS |
| NZ_LPHE01000134.1 | 8590 | 8772 | − | Burkholderia ubonensis | MSMB2092WGS |
| NZ_LPHF01000041.1 | 8590 | 8772 | − | Burkholderia ubonensis | MSMB2093WGS |
| NZ_LPJF01000006.1 | 63332 | 63514 | + | Burkholderia ubonensis | MSMB2207WGS |

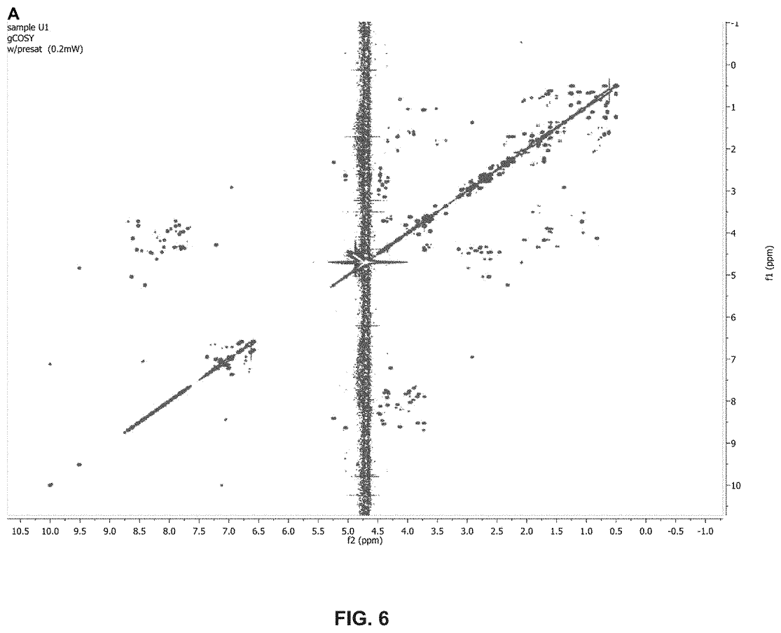

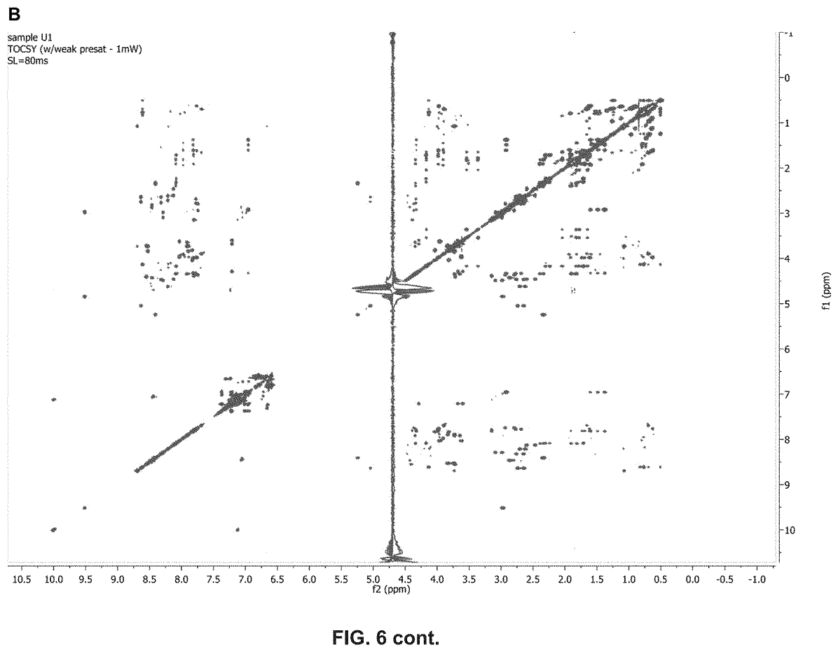

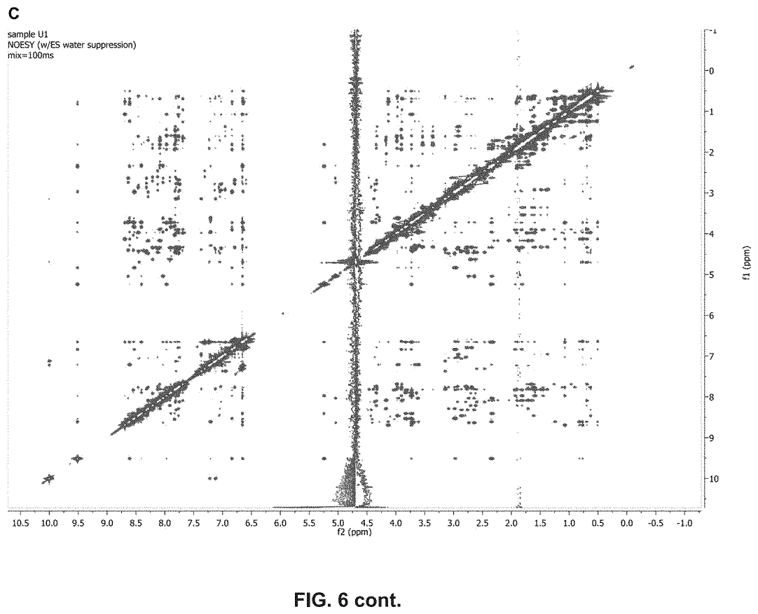

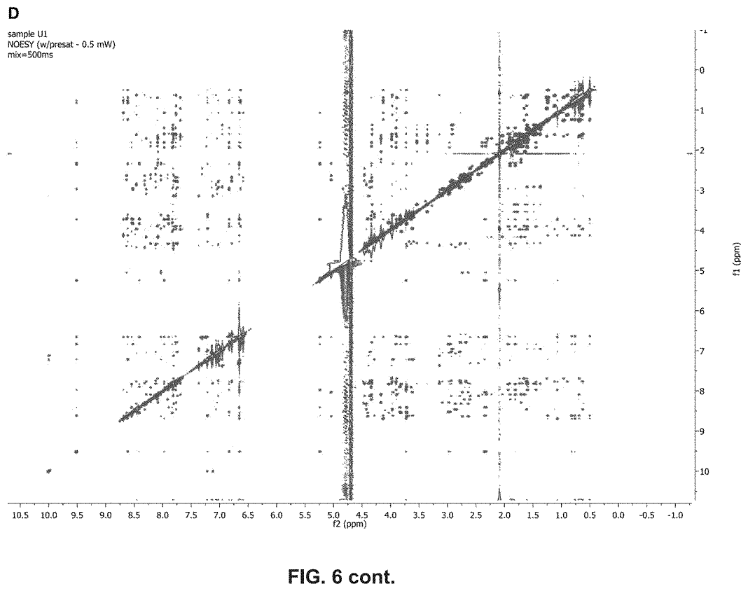

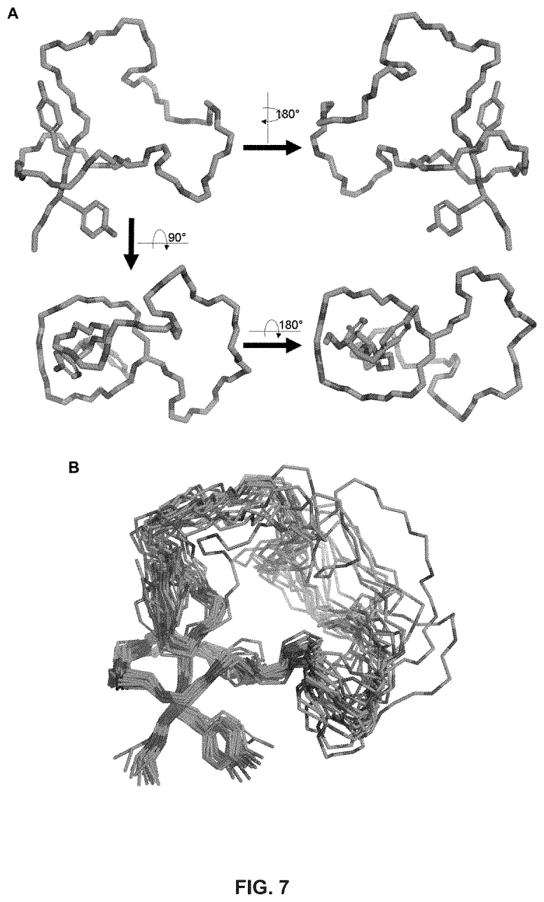

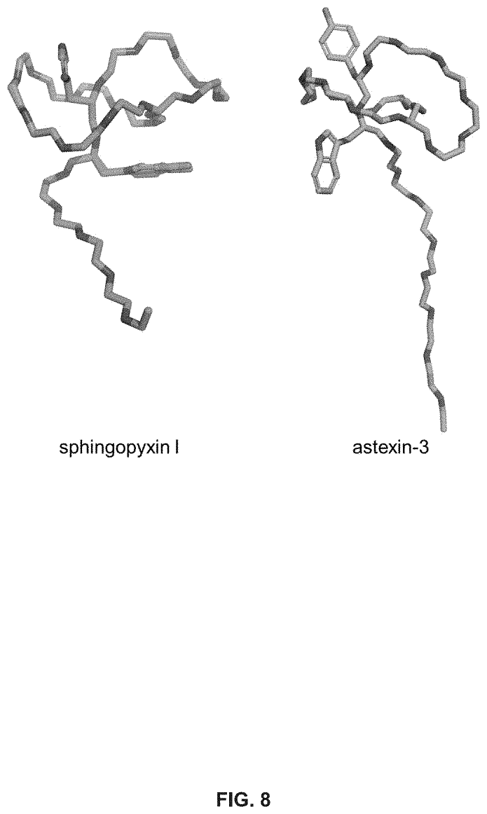

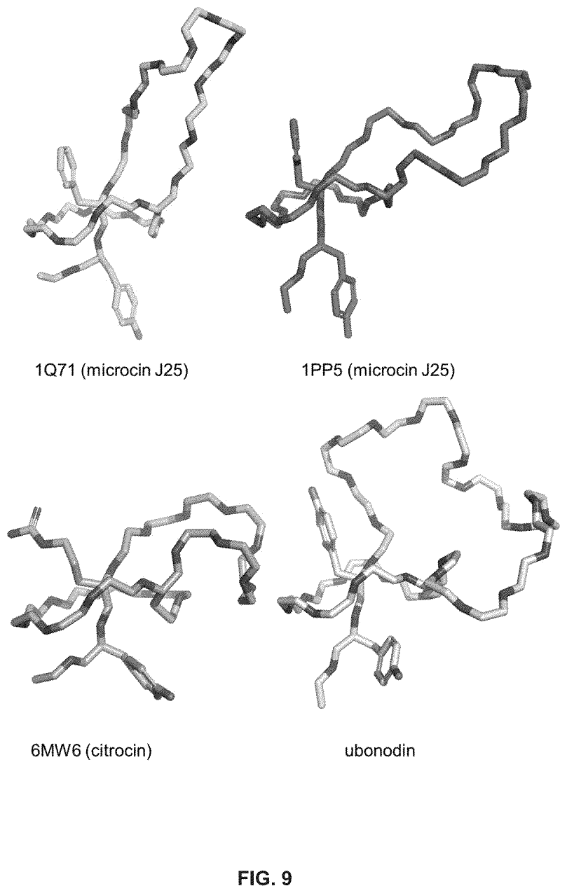

The structure of ubonodin was determined using 2D NMR experiments. A NOESY experiment was initially carried out with a long mixing time (500 ms) in order to assign all peaks along with COSY and TOCSY spectra. NOESY spectra were also acquired at shorter mixing times of 100 ms and 40 ms, with the 100 ms spectrum used for calculation of distance restraints (FIG. 6, Table S2, Table S3). Structure calculations revealed an unprecedented topology for a lasso peptide with an 8 aa isopeptide-bonded ring, an 18 aa loop, and a short 2 aa tail (FIG. 1; FIG. 7). Previously, the largest loop region observed in a lasso peptide with 10 aa was in microcin J25 (MccJ25). Other large protetobacterial lasso peptides such as astexin-3 (24 aa) and sphingopyxin I (26 aa), are characterized by relatively short loop regions (5 aa for astexin-3 and 6 aa for sphingopyxin I) and longer C-terminal tails (FIG. 8). Lasso peptides are often maintained in their [1]rotaxane structures by bulky steric lock residues that straddle the ring. In ubonodin, those residues are Tyr-26 and Tyr-27. This arrangement of steric lock residues is reminiscent of MccJ25 which uses Phe-19 and Tyr-20 as steric locks (FIG. 9). The large 18 aa loop of ubonodin is its most compelling structural feature. The ubonodin NOESY spectrum includes strong amide-amide crosspeaks indicative of turns. The most prominent turn in the loop runs from His-15 to Trp-19, a mostly polar stretch of the peptide with sequence HIHDW. Strong crosspeaks between sidechain resonances for Ile-16 and Trp-19 support the presence of this turn. There is also a shorter turn present that runs from Met-22 to Ser-24.

TABLE S2
Chemical shift assignments for ubonodin

| Residue | Hydrogen | Chemical Shift δ (ppm) |
|---|---|---|
| GLY-1 | H | 7.834 |
| GLY-1 | HA2 | 4.033 |
| GLY-1 | HA3 | 3.817 |
| GLY-2 | H | 8.545 |
| GLY-2 | HA2 | 3.719 |
| GLY-2 | HA3 | 4.407 |
| ASP-3 | H | 8.635 |
| ASP-3 | HA | 5.043 |
| ASP-3 | HB2 | 2.742 |
| ASP-3 | HB3 | 2.639 |
| GLY-4 | H | 8.033 |
| GLY-4 | HA2 | 3.959 |
| GLY-4 | HA3 | 3.623 |
| SER-5 | H | 7.208 |
| SER-5 | HA | 4.287 |
| SER-5 | HB2 | 3.676 |
| SER-5 | HB3 | 3.615 |
| ILE-6 | H | 8.609 |
| ILE-6 | HA | 4.131 |
| ILE-6 | HB | 0.824 |
| ILE-6 | QG2 | 0.768 |
| ILE-6 | HG12 | 1.243 |
| ILE-6 | HG13 | 0.694 |
| ILE-6 | QD1 | 0.506 |
| ALA-7 | H | 8.692 |
| ALA-7 | HA | 3.73 |
| ALA-7 | QB | 1.074 |
| GLU-8 | H | 7.974 |
| GLU-8 | HA | 3.904 |
| GLU-8 | HB2 | 1.598 |
| GLU-8 | HB3 | 1.447 |
| GLU-8 | HG2 | 1.935 |
| GLU-8 | HG3 | 1.815 |
| TYR-9 | H | 7.773 |
| TYR-9 | HA | 4.336 |
| TYR-9 | HB2 | 2.775 |
| TYR-9 | HB3 | 2.683 |
| TYR-9 | QD | 6.783 |
| TYR-9 | QE | 6.59 |
| PHE-10 | H | 7.743 |
| PHE-10 | HA | 4.366 |
| PHE-10 | HB2 | 2.936 |
| PHE-10 | HB3 | 2.786 |
| PHE-10 | QD | 7.024 |
| PHE-10 | QE | 7.138 |
| PHE-10 | HZ | 7.065 |
| ASN-11 | H | 8.125 |
| ASN-11 | HA | 4.459 |
| ASN-11 | HB2 | 2.604 |
| ASN-11 | HB3 | 2.46 |
| ASN-11 | HD21 | 7.31 |
| ASN-11 | HD22 | 6.661 |
| ARG-12 | H | 7.818 |
| ARG-12 | HA | 4.328 |
| ARG-12 | HB2 | 1.604 |
| ARG-12 | HB3 | 1.486 |
| ARG-12 | QG | 1.376 |
| ARG-12 | QD | 2.921 |
| ARG-12 | HE | 6.954 |
| PRO-13 | HA | 4.168 |
| PRO-13 | HB2 | 2.041 |
| PRO-13 | HB3 | 1.645 |
| PRO-13 | HG2 | 1.833 |
| PRO-13 | HG3 | 1.776 |
| PRO-13 | HD2 | 3.538 |
| PRO-13 | HD3 | 3.359 |
| MET-14 | H | 8.083 |
| MET-14 | HA | 4.169 |
| MET-14 | QB | 1.711 |
| MET-14 | HG2 | 2.318 |
| MET-14 | HG3 | 2.233 |
| HIS-15 | H | 8.288 |
| HIS-15 | HA | 4.484 |
| HIS-15 | HB2 | 3.093 |
| HIS-15 | HB3 | 2.979 |
| HIS-15 | HD2 | 7.045 |
| ILE-16 | H | 7.762 |
| ILE-16 | HA | 3.974 |
| ILE-16 | HB | 1.614 |
| ILE-16 | QG2 | 0.626 |
| ILE-16 | HG12 | 1.124 |
| ILE-16 | HG13 | 0.9 |
| ILE-16 | QD1 | 0.647 |
| HIS-17 | H | 8.46 |
| HIS-17 | HA | 4.427 |
| HIS-17 | QB | 2.862 |
| HIS-17 | HD2 | 7.066 |
| ASP-18 | H | 8.32 |
| ASP-18 | HA | 4.466 |
| ASP-18 | HB2 | 2.764 |
| ASP-18 | HB3 | 2.642 |
| TRP-19 | H | 7.809 |
| TRP-19 | HA | 4.39 |
| TRP-19 | QB | 3.146 |
| TRP-19 | HD1 | 7.123 |
| TRP-19 | HE3 | 7.371 |
| TRP-19 | HE1 | 10.002 |
| TRP-19 | HZ3 | 6.955 |
| TRP-19 | HZ2 | 7.219 |
| TRP-19 | HH2 | 7.01 |
| GLN-20 | H | 7.811 |
| GLN-20 | HA | 3.983 |
| GLN-20 | HB2 | 1.611 |
| GLN-20 | HB3 | 1.731 |
| GLN-20 | HG2 | 1.908 |
| GLN-20 | HG3 | 1.694 |
| ILE-21 | H | 7.689 |
| ILE-21 | HA | 3.897 |
| ILE-21 | HB | 1.657 |
| ILE-21 | QG2 | 0.7 |
| ILE-21 | HG12 | 1.26 |
| ILE-21 | HG13 | 0.963 |
| ILE-21 | QD1 | 0.685 |
| MET-22 | H | 8.09 |
| MET-22 | HA | 4.338 |
| MET-22 | HB2 | 1.903 |
| MET-22 | HB3 | 1.812 |
| MET-22 | HG2 | 2.394 |
| MET-22 | HG3 | 2.326 |
| ASP-23 | H | 8.213 |
| ASP-23 | HA | 4.629 |
| ASP-23 | HB2 | 2.709 |
| ASP-23 | HB3 | 2.614 |
| SER-24 | H | 7.913 |
| SER-24 | HA | 4.344 |
| SER-24 | HB2 | 3.73 |
| SER-24 | HB3 | 3.641 |
| GLY-25 | H | 7.896 |
| GLY-25 | HA2 | 3.837 |
| GLY-25 | HA3 | 3.726 |
| TYR-26 | H | 8.406 |
| TYR-26 | HA | 5.241 |
| TYR-26 | QB | 2.332 |
| TYR-26 | QD | 6.652 |
| TYR-26 | QE | 7.234 |
| TYR-27 | H | 9.512 |
| TYR-27 | HA | 4.835 |
| TYR-27 | QB | 2.971 |
| TYR-27 | QD | 6.832 |
| TYR-27 | QE | 6.638 |
| GLY-28 | H | 8.527 |
| GLY-28 | HA2 | 3.837 |
| GLY-28 | HA3 | 3.728 |

TABLE S3
Statistics for the ubonodin NMR structure calculations

| Constraints | Constraint Violations |
|---|---|
| Total = 400 | Distance violations, >0.5 Å: 0 |
| Distance, i = j: 103 | RMS deviations: 0.015 Å |
| Distance, \|i − j\| = 1: 145 | Average backbone RMSD to mean: 0.92 Å |
| Distance, \|i − j\| > 1: 152 | Average heavy atom RMSD to mean: 1.61 Å |

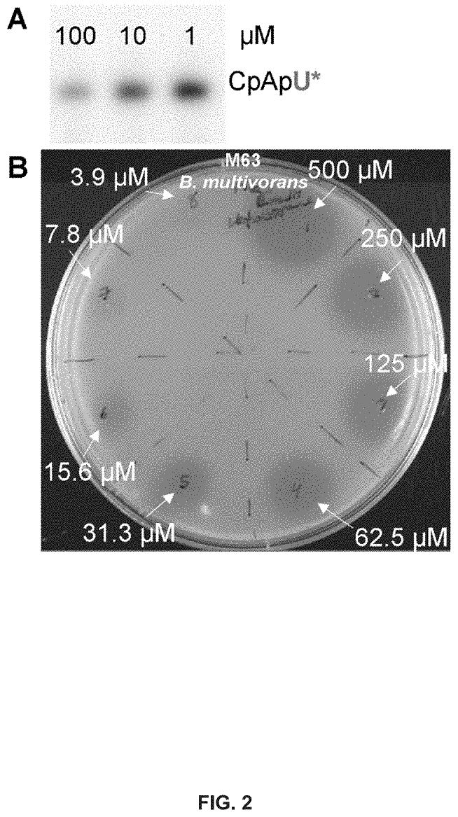

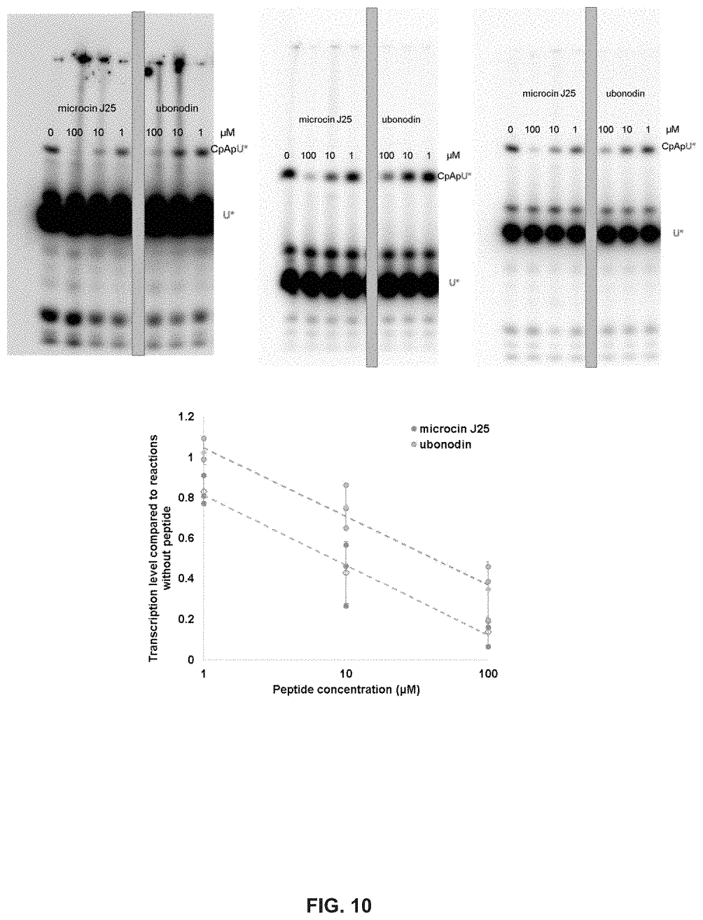

Given the similarity of the ring and tail portions of ubonodin to those of MccJ25 and citrocin, both of which exert their antimicrobial activity via inhibition of RNA polymerase (RNAP)^{14, 17} (FIG. 9), it was hypothesized that ubonodin would also function as an RNAP inhibitor. Abortive transcription initiation assays were carried out with E. coli RNAP (FIG. 2A). These assays confirmed that ubonodin inhibits transcription initiation, an activity and putative antimicrobial mode of action observed in several other lasso peptides.^{14-15, 18-20} The potency of ubonodin in these assays was somewhat lower than that of MccJ25 (FIG. 10), though this may be due to the fact that E. coli is not an antimicrobial target of ubonodin.

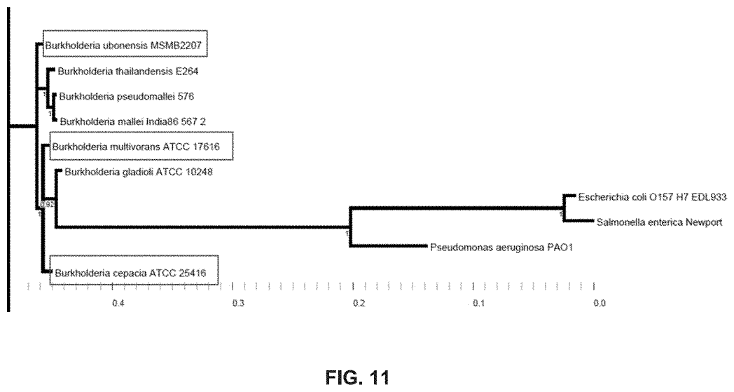

Encouraged by the RNAP-inhibiting activity of ubonodin, ubonodin was tested for antimicrobial activity against a panel of proteobacteria (Table 1, Table S4). Antimicrobial lasso peptides tend to have a narrow spectrum of activity, killing bacteria that are closely phylogenetically related. Ubonodin was unable to kill E. coli and Salmonella newport, strains that are susceptible to MccJ25 and citrocin. Given that ubonodin is encoded in the genome of a Burkholderia strain, ubonodin was tested against other Burkholderia. Modest activity of ubonodin was observed against the producing strain of the lasso peptide capistruin, B. thailandensis, and no activity against the plant pathogen B. gladiolii. The putative ubonodin producing strain, B. ubonenesis, belongs to the Burkholderia cepacia complex (Bcc), and potent activity was observed against two Bcc strains, B. multivorans and B. cepacia. These notorious strains are frequently found in lung infections in cystic fibrosis patients.²¹⁻²² In spot-on-lawn assays, these organisms were inhibited by low micromolar concentrations of ubonodin. The potency of ubonodin was affected by the media composition. For B. multivorans, the last active dilution in spot assays carried out in minimal M63 medium was 8 μM, whereas this increased to 20 μM on plates comprised of rich Mueller-Hinton medium. The spot-on-lawn were followed up by liquid growth assays in which the minimal inhibitory concentration of ubonodin was 4 μM against B. cepacia, and 31 μM against B. multivorans. The antimicrobial activity of ubonodin was also tested against the select agents B. pseudomallei and B. mallei. Ubonodin was also tested against two attenuated (BSL-2) strains of B. pseudomallei, Bp82 and Bp576 mn.²³⁻²⁴ While no activity was observed against any B. pseudomallei strains, growth inhibition of two B. mallei strains by ubonodin was observed in spot assays. Ubonodin has potent activity against Bcc strains with some activity against strains in the pseudomallei/mallei group (FIG. 11).

The structure and activity of ubonodin was also studied upon heating to 50° C. and 95° C. While ubonodin maintains its activity after heating to 50° C., it fragments into a variety of different structures and loses activity when heated to 95° C. (SI Text).

TABLE 1
Minimum inhibitory concentration (MIC) of ubonodin against
Burkholderia strains in Mueller-Hinton medium.

| | MIC via | MIC via |
|---|---|---|
| | spot-on-lawn | liquid growth |
| Strain | assay (μM) | assay (μM) |
| B. cepacia ATCC 25416 | 40 | 4 |
| B. multivorans ATCC 17616 | 20 | 31 |
| B. mallei Old ISU | 40 | >31 |
| B. mallei NVSL 86-567-2 | 40 | >31 |

TABLE S4
Antimicrobial activity of ubonodin. See also Table 1.

| Strain | MIC in M63 agar (μM) |
|---|---|
| Burkholderia multivorans ATCC 17616 | 8 |
| Burkholderia thailandensis E264 | 500 |
| Burkholderia gladioli ATCC 10248 | >500 |
| Burkholderia pseudomallei Bp82 | >500 |
| Burkholderia pseudomallei 576mn | >500 |
| Enterohemorrhagic Escherichia coli | >500 |
| O157:H7 TUV-93-0 | |
| Salmonella enterica serovar newport | >500 |
| Pseudomonas aeruginosa PAO1 | >500 |

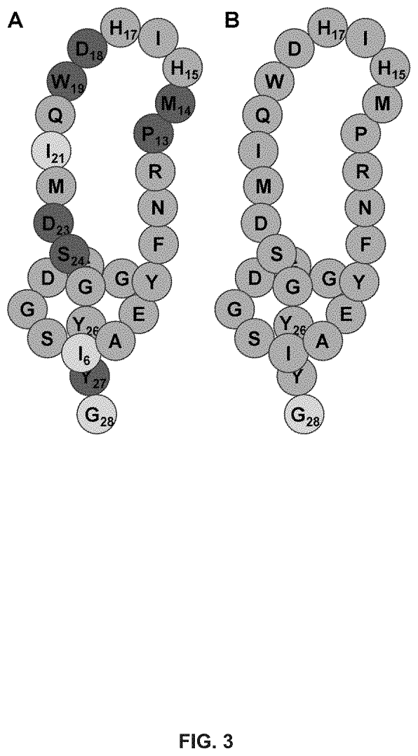

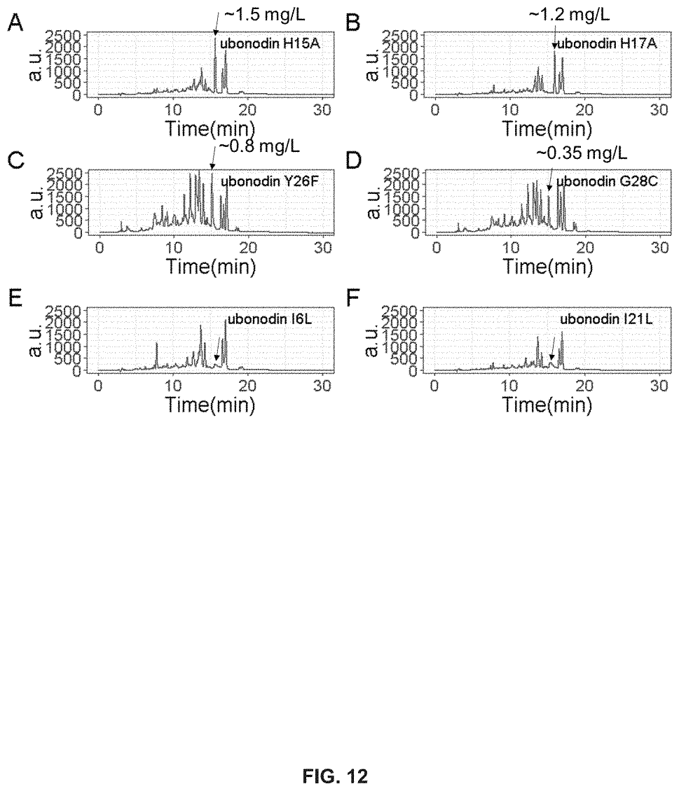

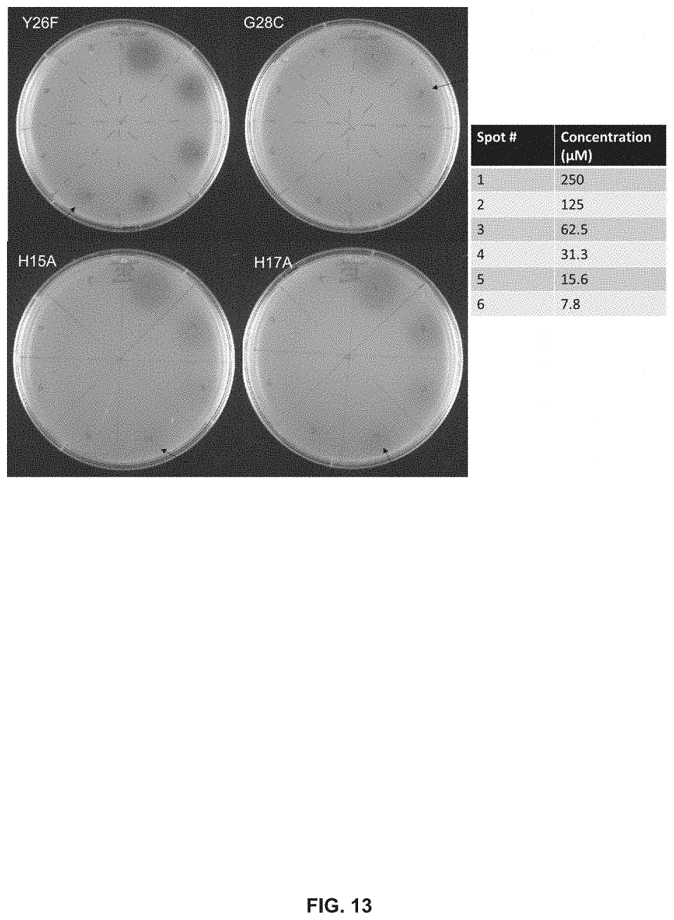

Mutagenesis on ubonodin was also performed to identify residues important for production and activity (FIG. 3). It was planned to utilize ubonodin as a starting material for peptide catenanes analogous to the MccJ25 catenanes described previously.²⁵ Therefore, Cys residues were introduced at the Pro-13, Met-14, and Trp-19 positions of ubonodin, as well as at the C-terminus of ubonodin, Gly-28. While all four of these variants were detected by LC-MS, only the G28C variant was produced at a quantity sufficient for purification (FIG. 12). This variant retained some antimicrobial activity against B. multivorans, but its activity was diminished relative to the wild-type peptide (FIG. 13). Mutagenesis was also carried out on the two steric lock residues, Tyr-26 and Tyr-27. While the Y26F variant of ubonodin was produced at roughly half of the wild-type level and retained antimicrobial activity, the Y27F variant was produced at levels only detectable by LC-MS. Substitution of either His residue (His-15 or His-17) with Ala surprisingly led to variants that expressed at near wild-type level and retained near wild-type antimicrobial activity against B. multivorans. This result suggests that these solvent exposed His residues are not critical for antimicrobial activity. Finally, a series of variants of ubonodin with conservative substitutions were generated: I6L, D18N, I21L, D23N, and S24A. While all of these variants were detected by LC-MS in crude culture supernatant extracts, only the I6L and I21L variants were detected as a unique peak on HPLC (FIG. 12). However, the peaks for the I6L and I21L variants of ubonodin were quite broad, suggesting that they may not exist as single defined structures. The NMR structure suggests that the 16 sidechain packs against the Y27 sidechain, thus switching 16 to Leu may disrupt the fold of ubonodin. While other lasso peptides are tolerant to amino acid substitutions,²⁶⁻²⁸ ubonodin appears to be fairly recalcitrant to mutagenesis.

Using genome mining and heterologous expression, a new antimicrobial lasso peptide, ubonodin, disclosed herein, was identified. Ubonodin exhibits potent antimicrobial activity against several strains of Burkholderia, including B. cepacia and B. multivorans, two Burkholderia pathogens that commonly cause infections in cystic fibrosis patients.²² It is shown that ubonodin is able to inhibit E. coli RNAP, suggesting that RNAP is the antimicrobial target of ubonodin. While ubonodin has activity against B. cepacia, B. muhtvorans, and B. mallei, it is poorly active against B. thailandensis and has no activity against B. gladiolii and B. pseudomallei. This narrow spectrum of activity may allow for therapeutic usage of ubonodin since it will only kill the target pathogens while leaving the healthy microbiome unscathed. The spectrum of activity of ubonodin could be dictated by its uptake into susceptible bacteria. Though the sequences and structures of RNAP-inhibiting lasso peptides MccJ25, citrocin, and ubonodin differ greatly, each of these peptides include Tyr residues at position 9 and at the penultimate position of the sequence. The C-terminal Gly residue is also conserved in each of these peptides. This Tyr/Tyr/Gly motif is likely an excellent predictor of RNAP-inhibiting lasso peptides. The structure of ubonodin differs from any other characterized lasso peptide with an 18 aa-long loop region. While turns in this loop region were observed from the NMR structure calculations, structures of MccJ25 bound to RNAP and the outer membrane receptor FhuA²⁹⁻³⁰ show significant remodeling of the MccJ25 loop region when bound to these proteins. Similar or even more drastic changes to the ubonodin loop may occur when bound to its target(s).

Table S5
Sequences of gBlocks for construction of the refactored ubonodin gene
cluster

| gBlock | Sequence |
|---|---|
| gBlock1 | GCGTTTTTTATTGGTGAGAATCCAAGCTAGCCATCAATTAAGAAAAAAATTTAGCTTGTAGATAAATTCAGAAGTTTTATTATTCCAATTGAGTGTAAAGGCATAAC |
| | TACAGGAGGGAGTGTGCAAAATGCCGTATGCGCTGSGCCAGCATGCGCGTCTGGCGTGTTATGAAGATGATCTGATTATTCTGACCATTCGTGATAATCGTTTTC |
| | ATCTGATCAAAGATGTGAGCCGTGATGCGGTGGACGCGTTATATGAACCGATGGCGGGACAGCGTGGCGCAGGACTGCATGACGCGCTTCGTATTATGGGCGT |
| | GCTGGAAGAGAGTCGCGATCGTGCGGATATTCCGCCTGCGGGACTGCGTCCGAAAAGCTATGTGGAACAGCGTTGGATGATGCCGCTGACTCGGCATGCTCC |
| | GGCGACCTTAGTGGGCACCGTGGCGTCGCTGGTGGCACTGTATCGTGCAACCCTGATGATTAAACTGGGCGGCTTTCGTCGTATTGTGAGCATTGGCAAATGG |
| | CCGGCTCGTATGGCGAGCGGCAGTGTCGATGTGGATGGCACAGTGCAGGCTGCAATGGGCGATCTGAACCGTGTGTTTGCGTGCGATGTGTCTGGCAATCGT |
| | TGCCTGACCTATAGCCTGGCGCTGACCCTGCTGCTGCGTCGTAAAATTCCGAATGTTTCACTGGTGGTGGGCGTCCGTACCCGTCCGTTTTTTAGCCATGCGTG |
| | GGTTGAAGTGGACGGTCGCGTGGTGAATGATACCGCGGATCTGCGTAAAAATCTGGCGGTGATCCTGGAGGTTTGATGTTCATTGCCTACCCTGAGAACATAGC |
| | GAAGCATTTGGAATACATCATTGATGAATGGGCTGGTCGTAATCGTCTGACCACCCACCTGCATGGTAAGTTCGTGGTGCGTTGTAGCGATCGTTGGACCGTGA |
| | GCAAGTGGGGCAATTTCATTGAGTTCTTTGAAGGGATGGCTTATGAGTGGCCGACTGCCAGGCTTGGCCAGCGCCGGAGCGTCGTGCTCGTCTTAGTAATAG |
| | CATTGGCTATTTTACCAGCATTCTGTTGGATTCCGATACCCTGGAAGTCGTGCGCAGCCTGTATCGTGCGACCGAGATCTTTTATACCGAAAGCGATGGCATGAT |
| | GTTAGCGTGTAGTGAACTGGCTGTGGTGGTCGCGCTTCGTGGCGGATTTGCACGCCAGCGTATTGATGTGGATTATTGCCATGATTTCATAGCACACCAACAAA |
| | AGTTCGACGGACATACGTCATGTGAATCAATTAACGAGGTGATGCTGGGCGAATGTATACGGATGAGCACAAGCGATATCATTTCAGCTGCGTTTGTGA&TCGTC |
| | CGATTGTCCCGAGCGGCGACATCGTGGACACCTTGCGTGATACCTTAGCGGCATTTACACGTCCATTTGATGGCACCGTCCTGATGTTTAGCGGGGGTCTGGAt |
| | AGCAGTACCCTGTTGTGGACTCT |
| gBlock2 | GGATAGCAGTACCCTGTTGTGGACTCTGCTGGAATCTGGGACTAACCGCTGGTGCTGCACAGCGAGTCTGGGCCGGATGCGCGTGACAGCGAATACCAGGA |
| | CGCAGCGGCAGTGGCACTGGATCTGGGCTGCGAAATTCAGCGTTTCGTGCCGGGACGCGAGGACTATAGCCGCGCTTTTACTATCAGTGATGACGGCCAAAG |
| | CAGCAGTCCGTATGATATTCCCATCTTCCTGTCTCGTAGCTCTGCTCGTTCGGGCTTATCTATCGATGAAACCAGCCTGCTGGTGACCGGGCATGGGGGTGACC |
| | ATGTGTTCGTGCAGAACCCCGAAAACAACTCCTGCTTGGCGGCTCTTCAAGCGGGACGGGTGTTTGAGTATCTGCGTACGGTGCGTAAACTGAGCCGTCTGAA |
| | AGGCCGTCGGGGCGTGGAGATTGTACGGCATAACCTGCGCCTGCTTATGGGAGGCCATCTGCTGTCAGGTTCGTTCCCGGATTGGCTGCCGCGTCCGCGGCA |
| | CCGCTCCGCACGTCGGACAGGCCACTACTTAATTCGCGACCTGGACCGGCGTATGGCGAAACATACTCATCTGAGCGCCATTCTGCAGGCTTTACAGAGCGCA |
| | AGCATTCCGCGCAACGGACCGCCCATGTTGGCGCCGTTGTTGCTGCAAAATGTGATTGGCCATATGATGGGCATACCGGTTCAGGATACGTTTACTGAAACCCA |
| | TGATCGTGTGACACTTCGTGAGTCGATTTATCGTCAGTCTGGCAAATCTTTTGCGTGGCGTCGTACCAAACGTGCGTCCAGCGCTTTCCTGTTTGAACTGTTGAG |
| | CCAGTCCGAAGTGAATTTAGCCGATCTGATCGACCGCAGCCACTTTGTTCCACTGTTGCATATTGATCGTCGGGCATTACTGGCGGAAGTGCGTCAGAATTGCC |
| | GGATAGCGCTGACCGGCAACTTTAAACATATTGTCAACTTGTATAAGATTGAAGCGCACCTTCGCTCAATAGAGCATCAGTCTGCAGAACTAACCAGACCATGAA |
| | GCGTTGGATCGGTATCTATTCTGAGATCGGCCACCATTTGCAACGCCAGGAACGGTACTTTGTAGTAGCAATTCTGTTTTGCACCCTTGGTGCTGCGGCCAGCA |
| | TGGCGATGAGCCCGGTGTTTTTAGGGCGTCTGGCGGATTCACTGCTTGCGGCGGATCGTCGTATGCCCGCGTACATTATCTACTTAGCGGCAAGCTATTTGATC |
| | &CCATTGCTATGCCAAAGCTGCTGGGCACCGTAGATCTGTACCTGCAGTCAATGTTGCGTTTACGTGCGAACCGTAGCCTGTTAGCCGGGTACTTCAACTATCT |
| | GTGTCGGCAACCCGAGAGTTTTTTCGTGAATAAGAATAGTGGTGAGCTTACCCAAGAGATCACCCAAGCGTCTAATGATCTTTACCTGATTGTACGGAACCTGAC |
| | CACTAGCCTTATCTCGCCGATTGTGCAGGTGAG |
| gBlock3 | CTTATCTCGCCGATTCTGCAGGTGAGCATTGCGGTGGTCGTCCTTGCGAGCAATCATGACCTGTTGGTGGCGGGGACGATAGCGATTTATGTGGCTTTGTTCGT |
| | AACAAAGAATGTAATACATGGCCGTCGTTTGGTAGAACTGAAATTCCGTTGCATGGATGCAGGTCGGAASAGCTATGGAACGTTGACGGACAGCATCACDAATAT |
| | TCAGGTGGCGCGTCAGTTTAATGGGTATCGTTTCCTGTTGAGCCGCTATCAACGGGTGCTTGACGAAGACCGTCATACACAGGGCGACTACTGGAAGATCTCTC |
| | TGCGTATGCAGTTTTTCAACGCGTGTCTGTTTGTGGGCCTGTTTGGCGTAACCTTTCTGATGGCGCTGCACGAAGTAGTGACCGGTGCGCGCTCTATTGGCAAT |
| | TTCGTGCTTGTCGCCGCGTATACCGTGACCCTGTTAAGCCCCATCGAGATTCTGGGCAATATGTTTACCGAAATTAACCAGAGTCTGGTGACCTTTGGGCGTTTT |
| | CTTGATAAATTGTCAGCAGCCACAGCTCCTCTTAGCCAGCGTGCGCCTAAGCCGGCAGTTAAGTCCGCGGCACCGGCTATCGAATTTGAACGTGTGTGCGTGA |
| | CCTATCCGGGTGCCAATCGCCAGGCATTAACTGATGTGGGCTTTACAGTGGATGCCGGAAAGCGTGTAGCGATAACTGGTCCCTCTGGAGCAGGCAAGAGCAG |
| | CCTGGTGAAAGTTCTGACCCGCCAACTTGTGGCGGAAGAAGGAGCCATTCGTATTTTTGGCGAGGATATCTTATGTATTGATGCGCAGACCCTGAGCGAACGTA |
| | TTGGCTGCGTGTCACAGGACGTACTGCTGTTTAAAGATACCTTACGGTTTAACTTGCAGATTTCGCGCCCTGATGCTTCGGACGCGGACATGGTCACTGCACTT |
| | GAGTGCGCGGGACTGACGGATCTTTTAGTGGACTTACCTGCCGGGTTGGACACGATGTTAGGCGATCGTGGCGCAACACTTTCTGGAGGTCAGCGTCAGCGGT |
| | TGGCGTTAGCCCGCTTGTTCCTGCGTGCCCCCGACATTGTGTTGGTTGATGAAGGCACCTCTTCGCTGGATTTGGTAACTGAGCAGTATGTTCTGGACAAGGTG |
| | TTTGAAGTGTTTAGCGACAAAACCATAGTGATGATAACCCACCGTCCTAGCGCGATGADCAAAGTTGATGCCGTGATTATCATGAGCGATGGTCGTATTGACGAT |
| | CATGCGGAACCGGATGTGCTTCGTAGCCGTAATACCTTTTTTGCGCGTGTTGTGGAATCTTCTTTGCGTTGACCATGGGCAAATATTATACGCAAGGCGAC |

TABLE S6
Sequences of primers used in this study

| Primer Name | Sequence |
|---|---|
| bubA Asmbly F1 | AATAGCGAATTCATTAAAGAGGAGAAATTAACTATGAAAAATCGTAGCACCAAAG |
| bubA Asmbly R2 | CACATCGCCAATGCAGGTAATTTCGAAGCTCTCTTTGGTGCTACGATTTTTCATAG |
| bubA Asmbly F3 | ACCTGCATTGGCGATGTGGATGTGATTACCCTGATGCAGGATGCGAGCCGTGCGAC |
| bubA Asmbly R4 | GACGGTTAAAGTATTCCGCAATGCTGCCATCGCCTCCCATTGTCGCACGGCTCGCA |
| bubA Asmbly F5 | ATTGCGGAATACTTTAACCGTCCGATGCATATTCATGATTGGCAGATTATGGATAG |
| bubA Asmbly R6 | AGGTCAAGCTTTCAGCCATAATAGCCGCTATCCATAATCTGCCAATCATGAA |
| bub_cluster_SeqF1 | CAGTGTCGATGTGGATGGCA |
| bub_cluster_SeqF2 | CAACAAAAGTTCGACGGACATAC |
| bub_cluster_SeqF3 | TCAGGTTCGTTCCCGGATTG |
| bub_cluster_SeqF4 | GATCGTCGTATGCCCGCGTA |
| bub_cluster_SeqF5 | GCTCTATTGGCAATTTCGTG |
| uboA-EcoRI-For | AATAGCGAATTCATTAAAGAGGAGAAATTAACTATGAAAAATCGTAG |
| pQE-HindIII-Rev | CAACAGGAGTCCAAGCTCAGCTAATTAAG |
| uboA_I6L-For | GATGGCAGCCTGGCGGAATACTTTAACC |
| ubo_I6L-Rev | GGTTAAAGTATTCCGCCAGGCTGCCATC |
| uboA P13C For | GAATACTTTAACCGTTGCATGCATATTCATG |
| uboA P13C Rev | CATGAATATGCATGCAACGGTTAAAGTATTC |
| uboA M14C For | TACTTTAACCGTCCGTGCCATATTCATGATT |
| uboA M14C Rev | AATCATGAATATGGCACGGACGGTTAAAGTA |
| uboA_H15A-For | CTTTAACCGTCCGATGGCGATTCATGATTGG |
| uboA_H15A-Rev | CCAATCATGAATCGCCATCGGACGGTTAAAG |
| uboA-H17A-For | GTCCGATGCATATTGCGGATTGGCAG |
| uboA-H17A-Rev | CTGCCAATCCGCAATATGCATCGGAC |
| uboA D18N For | CGATGCATATTCATAATTGGCAGATTATG |
| uboA D18N Rev | CATAATCTGCCAATTATGAATATGCATCG |
| uboA D23N Rev | ACATTAAGCTTTCAGCCATAATAGCCGCTATTCATAATCTG |
| uboA S24A Rev | TGCTTAAGCTTTCAGCCATAATAGCCGGCATCCATAATCTG |
| uboA W19C For | ATGCATATTCATGATTGCCAGATTATGGATA |
| uboA W19C Rev | TATCCATAATCTGGCAATCATGAATATGCAT |
| ubo_I21L_Rev | GATTGGCAGCTGATGGATAGCGGCTATTATGGCTGAAAGCTTAAGAA |
| uboA G28C Rev | TAATTAAGCTTTCAGCAATAATAGCCGC |
| uboA Y26F Rev | TAATTAAGCTTTCAGCCATAAAAGCCGCTATC |
| uboA Y27F Rev | TAATTAAGCTTTCAGCCAAAATAGCCGCT |

TABLE S7
Bacterial strain information

| Bacterium | Strain | Biosafety Level |
|---|---|---|
| Burkholderia cepacia | ATCC 25416 | 2 |
| Burkholderia mallei | China 5 (NBL 4) | 3 |
| Burkholderia mallei | China 7 (NBL 7) | 3 |
| Burkholderia mallei | 85-503 | 3 |
| Burkholderia mallei | Old ISU | 3 |
| Burkholderia mallei | Turkey | 3 |
| Burkholderia mallei | NVSL 86-567-2 | 3 |
| Burkholderia pseudomallei | Human/Blood/OH/US/1994 | 3 |
| Burkholderia pseudomallei | 1710a | 3 |
| Burkholderia pseudomallei | K96243 | 3 |
| Burkholderia multivorans | ATCC 17616 | 2 |

Ubonodin Thermal Stability

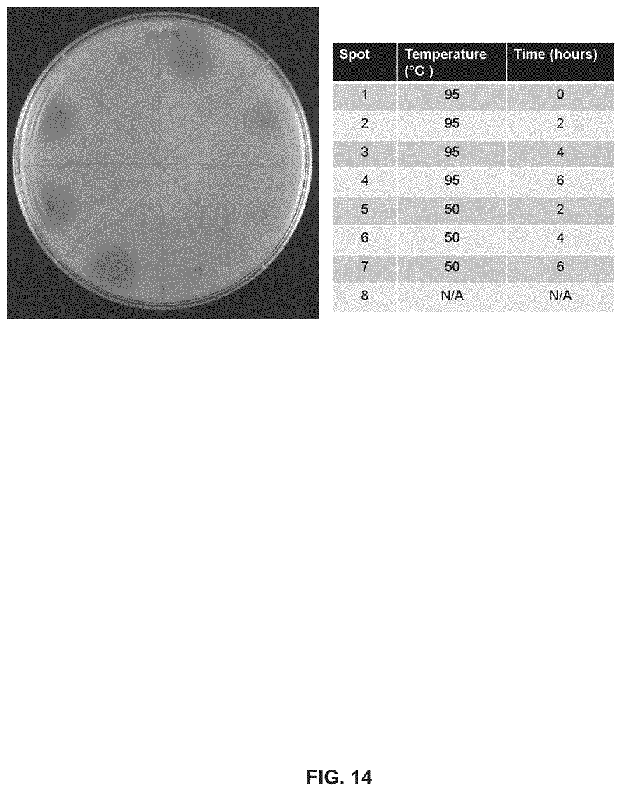

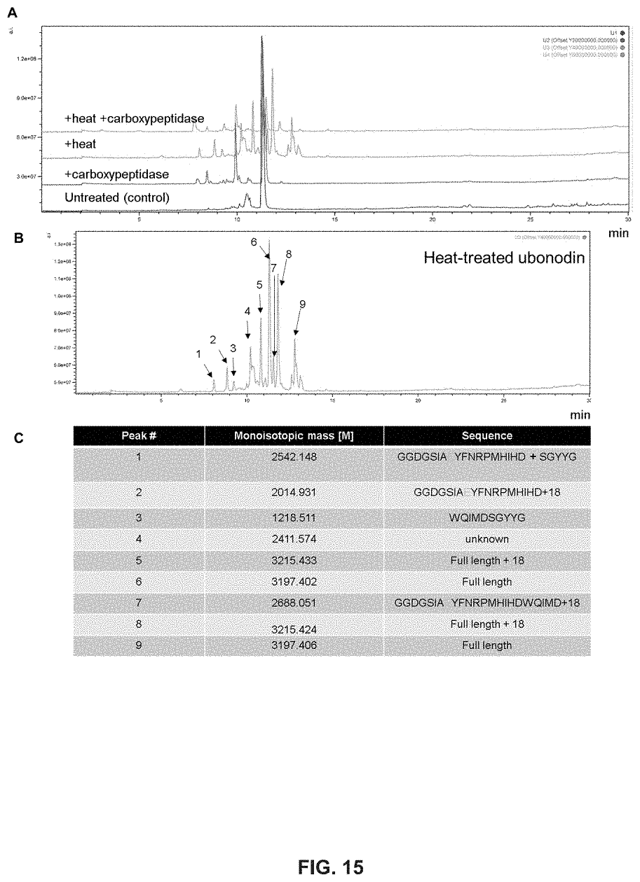

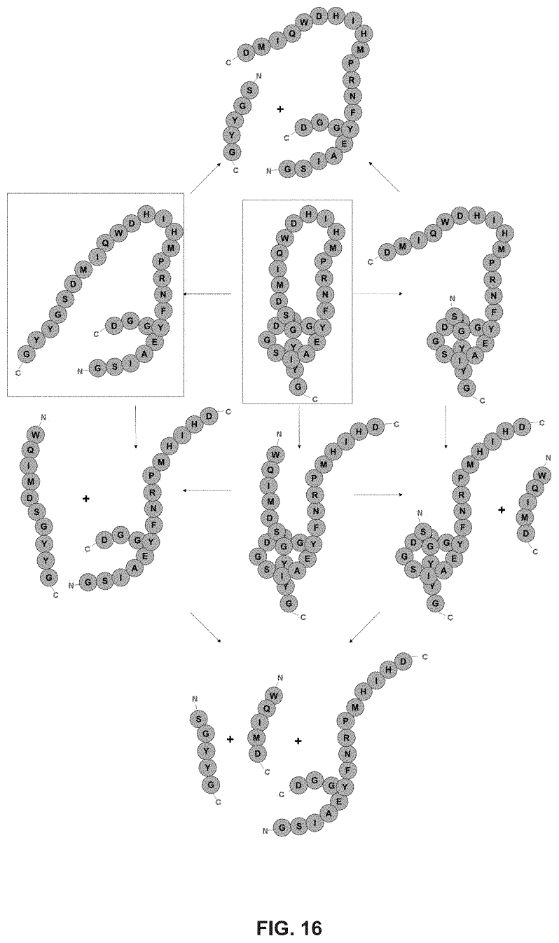

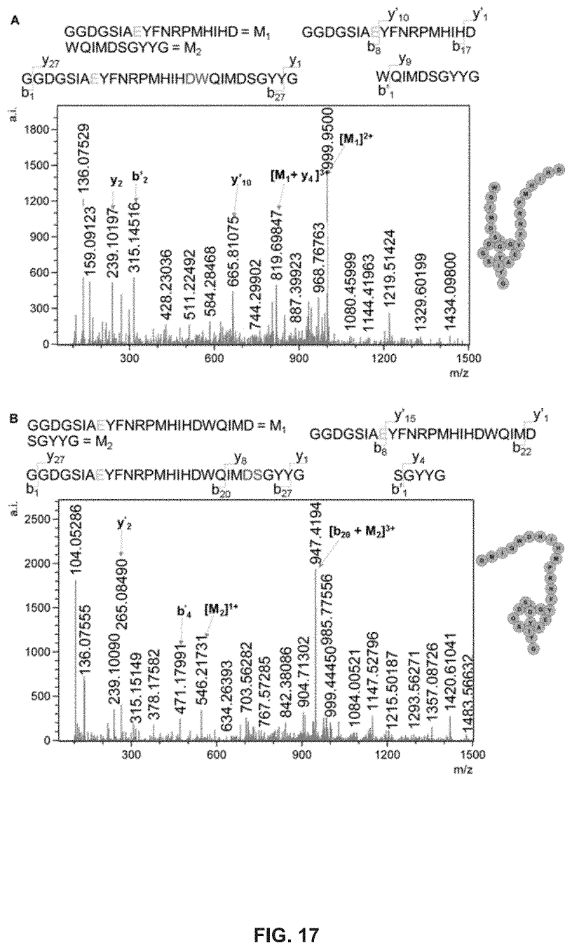

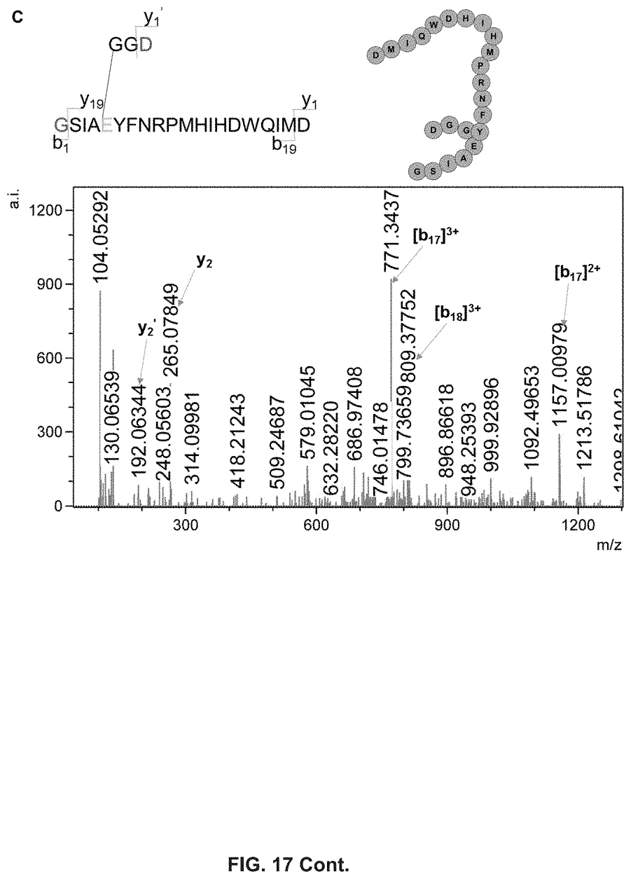

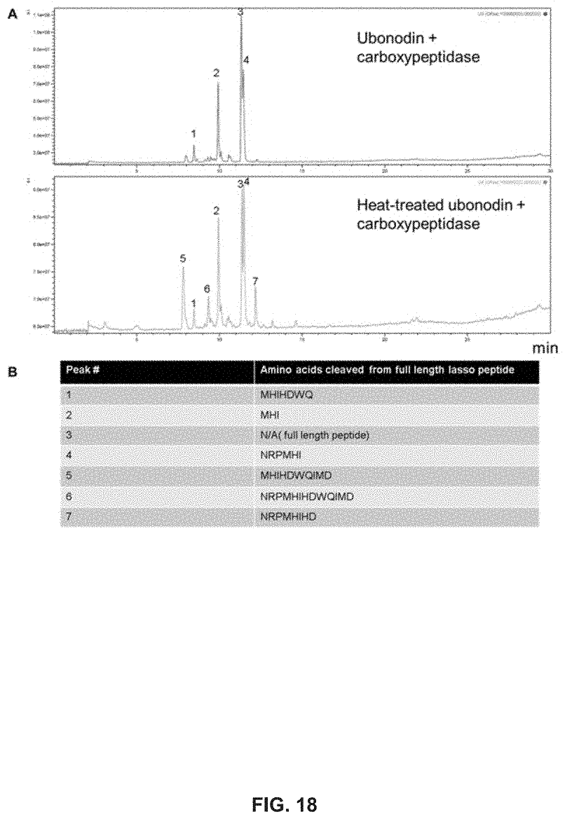

terminal to Asp residues has also been observed in lasso peptides.³⁸⁻³⁹ A sample of ubonodin was heated to either 50° C. or 95° C. for up to 6 h and observed that ubonodin retained activity against B. multivorans after heating to 50° C., but not to 95° C. (FIG. 14). Since a loss in activity can be due either to lasso peptide unthreadine or cleavage after Asp residues, a sample of ubonodin was next heated to 95° C. for 2 h by LC-MS². The major species in this sample is still intact ubonodin (FIG. 15). Several new peaks were observed and could assign many of them to cleavages of ubonodin C-terminal to each of the three Asp residues, two within the loop and one within the ring of ubonodin (FIGS. 16, 17). Peptides cleaved after the two loop Asp residues (Asp-18, Asp-23, or both) remain threaded, generating a series of [2]rotaxane structures. Further cleavage of heat-treated ubonodin with carboxypeptidase, which hydrolyzes amino acids with a free C-terminus, confirmed the assignment of the [2]rotaxane peptides (FIG. 17). Species were observed consistent with cleavage after Asp-3 in the ring of ubonodin plus an additional cleavage after either Asp-18 or Asp-23 residue in the loop (FIGS. 16, 17). There is an additional peak with mass identical to intact ubonodin, but with a different retention time. This peak could correspond to an unthreaded species of ubonodin as it is completely eliminated upon carboxypeptidase digestion (FIGS. 15, 18). The thermal degradation of ubonodin is unlike that of any other lasso peptide. Whereas most lasso peptides exhibit either thermostability or unthreading, ubonodin, due to the presence of multiple Asp residues, “self-destructs” into a variety of different peptide fragments.

### REFERENCES

(1) Rhodes, K. A.; Schweizer, H. P., Antibiotic resistance in Burkholderia species. Drug resistance updates: reviews and commentaries in antimicrobial and anticancer chemotherapy 2016, 28, 82-90.
(2) Hatcher, C. L.; Muruato, L. A.; Torres, A. G., Recent Advances in Burkholderia mallei and B. pseudomallei Research. Current tropical medicine reports 2015, 2 (2), 62-69.
(3) Sousa, S. A.; Ramos, C. G.; Leitão, J. H., Burkholderia cepacia Complex: Emerging Multihost Pathogens Equipped with a Wide Range of Virulence Factors and Determinants. Int. J. Microbiol. 2011, 2011, 607575.
(4) Mahenthiralingam, E.; Urban, T. A.; Goldberg, J. B., The multifarious, multireplicon Burkholderia cepacia complex. Nature Reviews Microbiology 2005, 3, 144.
(5) Diaz Caballero, J.; Clark, S. T.; Wang, P. W.; Donaldson, S. L.; Coburn, B.; Tullis, D. E.; Yau, Y. C. W.; Waters, V. J.; Hwang, D. M.; Guttman, D. S., A genome-wide association analysis reveals a potential role for recombination in the evolution of antimicrobial resistance in Burkholderia multivorans. PLoS Path. 2018, 14 (12), e1007453.
(6) Biggins, J. B.; Liu, X. F.; Feng, Z. Y.; Brady, S. F., Metabolites from the Induced Expression of Cryptic Single Operons Found in the Genome of Burkholderia pseudomallei. J. Am. Chem. Soc. 2011, 133 (6), 1638-1641.
(7) Mao, D. N.; Bushin, L. B.; Moon, K.; Wu, Y. H.; Seyedsayamdost, M. R., Discovery of scmR as a global regulator of secondary metabolism and virulence in Burkholderia thailandensis E264. Proc. Natl. Acad. Sci. U.S.A 2017, 114 (14), E2920-E2928.
(8) Arnison, P. G.; Bibb, M. J.; Bierbaum, G.; Bowers, A. A.; Bugni, T. S.; Bulaj, G.; Camarero, J. A.; Campopiano, D. J.; Challis, G. L.; Clardy, J.; Cotter, P. D.; Craik, D. J.; Dawson, M.; Dittmann, E.; Donadio, S.; Dorrestein, P. C.; Entian, K.-D.; Fischbach, M. A.; Garavelli, J. S.; Goransson, U.; Gruber, C. W.; Haft, D. H.; Hemscheidt, T. K.; Hertweck, C.; Hill, C.; Horswill, A. R.; Jaspars, M.; Kelly, W. L.; Klinman, J. P.; Kuipers, O. P.; Link, A. J.; Liu, W.; Marahiel, M. A.; Mitchell, D. A.; Moll, G. N.; Moore, B. S.; Muller, R.; Nair, S. K.; Nes, I. F.; Norris, G. E.; Olivera, B. M.; Onaka, H.; Patchett, M. L.; Piel, J.; Reaney, M. J. T.; Rebuffat, S.; Ross, R. P.; Sahl, H.-G.; Schmidt, E. W.; Selsted, M. E.; Severinov, K.; Shen, B.; Sivonen, K.; Smith, L.; Stein, T.; Sussmuth, R. D.; Tagg, J. R.; Tang, G.-L.; Truman, A. W.; Vederas, J. C.; Walsh, C. T.; Walton, J. D.; Wenzel, S. C.; Willey, J. M.; van der Donk, W. A., Ribosomally synthesized and post-translationally modified peptide natural products: overview and recommendations for a universal nomenclature. Nat. Prod. Rep. 2013, 30 (1), 108-160.
(9) Maksimov, M. O.; Pan, S. J.; Link, A. J., Lasso peptides: structure, function, biosynthesis, and engineering. Nat. Prod. Rep. 2012, 29, 996-1006.
(10) Hegemann, J. D.; Zimmermann, M.; Xie, X.; Marahiel, M. A., Lasso Peptides: An Intriguing Class of Bacterial Natural Products. Acc. Chem. Res. 2015, 48 (7), 1909-1919.
(11) Knappe, T. A.; Linne, U.; Zirah, S.; Rebuffat, S.; Xie, X. L.; Marahiel, M. A., Isolation and structural characterization of capistruin, a lasso peptide predicted from the genome sequence of Burkholderia thailandensis E264. J. Am. Chem. Soc. 2008, 130 (34), 11446-11454.
(12) Maksimov, M. O.; Pelczer, I.; Link, A. J., Precursor-centric genome-mining approach for lasso peptide discovery. Proc. Natl. Acad. Sci. U.S.A 2012, 109 (38), 15223-15228.
(13) Maksimov, M. O.; Link, A. J., Prospecting genomes for lasso peptides. J. Ind. Microbiol. Biotechnol. 2014, 41 (2), 333-344.
(14) Cheung-Lee, W. L.; Parry, M. E.; Cartagena, A. J.; Darst, S. A.; Link, A. J., Discovery and structure of the antimicrobial lasso peptide citrocin. J. Biol. Chem. 2019, 294 (17), 6822-6830.
(15) Wilson, K. A.; Kalkum, M.; Ottesen, J.; Yuzenkova, J.; Chait, B. T.; Landick, R.; Muir, T.; Severinov, K.; Darst, S. A., Structure of microcin J25, a peptide inhibitor of bacterial RNA polymerase, is a lassoed tail. J. Am. Chem. Soc. 2003, 125 (41), 12475-12483.
(16) Zirah, S.; Afonso, C.; Linne, U.; Knappe, T. A.; Marahiel, M. A.; Rebuffat, S.; Tabet, J. C., Topoisomer Differentiation of Molecular Knots by FTICR MS: Lessons from Class II Lasso Peptides. J. Am. Soc. Mass Spectrom. 2011, 22 (3), 467-479.
(17) Delgado, M. A.; Rintoul, M. R.; Farias, R. N.; Salomon, R. A., Escherichia coli RNA polymerase is the target of the cyclopeptide antibiotic microcin J25. J. Bacteriol. 2001, 183 (15), 4543-4550.
(18) Mukhopadhyay, J.; Sineva, E.; Knight, J.; Levy, R. M.; Ebright, R. H., Antibacterial peptide microcin J25 inhibits transcription by binding within and obstructing the RNA polymerase secondary channel. Mol. Cell 2004, 14 (6), 739-751.
(19) Kuznedelov, K.; Semenova, E.; Knappe, T. A.; Mukhamedyarov, D.; Srivastava, A.; Chatterjee, S.; Ebright, R. H.; Marahiel, M. A.; Severinov, K., The Antibacterial Threaded-lasso Peptide Capistruin Inhibits Bacterial RNA Polymerase. J. Mol. Biol. 2011, 412, 842-848.
(20) Metelev, M.; Arseniev, A.; Bushin, L. B.; Kuznedelov, K.; Artamonova, T. O.; Kondratenko, R.; Khodorkovskii, M.; Seyedsayamdost, M. R.; Severinov, K., Acinetodin and Klebsidin, RNA Polymerase Targeting Lasso Peptides Produced by Human Isolates of Acinetobacter gyllenbergii and Klebsiella pneumoniae. Acs Chemical Biology 2017, 12 (3), 814-824.
(21) Vandamme, P.; Holmes, B.; Vancanneyt, M.; Coenye, T.; Hoste, B.; Coopman, R.; Revets, H.; Lauwers, S.; Gillis, M.; Kersters, K.; Govan, J. R. W., Occurrence of multiple genomovars of Burkholderia cepacia in cystic fibrosis patients and proposal of Burkholderia multivorans sp. nov. Int. J. Syst. Bacteriol. 1997, 47 (4), 1188-1200.
(22) LiPuma, J. J., The Changing Microbial Epidemiology in Cystic Fibrosis. Clin. Microbiol. Rev. 2010, 23 (2), 299-323.
(23) Propst, K. L.; Mima, T.; Choi, K. H.; Dow, S. W.; Schweizer, H. P., A Burkholderia pseudomallei Delta purM Mutant Is Avirulent in Immunocompetent and Immunodeficient Animals: Candidate Strain for Exclusion from Select-Agent Lists. Infect. Immun. 2010, 78 (7), 3136-3143.
(24) Norris, M. H.; Khan, M. S. R.; Schweizer, H. P.; Tuanyok, A., An avirulent Burkholderia pseudomallei Delta purM strain with atypical type B LPS: expansion of the toolkit for biosafe studies of melioidosis. BMC Microbiol. 2017, 17, 1-14.
(25) Allen, C. D.; Link, A. J., Self-Assembly of Catenanes from Lasso Peptides. J. Am. Chem. Soc. 2016, 138 (43), 14214-14217.
(26) Pavlova, O.; Mukhopadhyay, J.; Sineva, E.; Ebright, R. H.; Severinov, K., Systematic structure-activity analysis of microcin J25. J. Biol. Chem. 2008, 283 (37), 25589-25595.
(27) Pan, S. J.; Link, A. J., Sequence Diversity in the Lasso Peptide Framework: Discovery of Functional Microcin J25 Variants with Multiple Amino Acid Substitutions. J. Am. Chem. Soc. 2011, 133, 5016-5023.
(28) Maksimov, M. O.; Koos, J. D.; Zong, C.; Lisko, B.; Link, A. J., Elucidating the Specificity Determinants of the AtxE2 Lasso Peptide Isopeptidase. J. Biol. Chem. 2015, 290 (52), 30806-30812.
(29) Braffman, N.; Piscotta, F. J.; Hauver, J.; Campbell, E. A.; Link, A. J.; Darst, S. A., Structural mechanism of transcription inhibition by lasso peptides microcin J25 and capistruin. Proc. Natl. Acad. Sci. U.S.A 2019, 116, 1273-1278.
(30) Mathavan, I.; Zirah, S.; Mehmood, S.; Choudhury, H. G.; Goulard, C.; Li, Y.; Robinson, C. V.; Rebuffat, S.; Beis, K., Structural basis for hijacking siderophore receptors by antimicrobial lasso peptides. Nat Chem Biol 2014, 10 (5), 340-342.
(31) Hoover, D. M.; Lubkowski, J., DNAWorks: an automated method for designing oligonucleotides for PCR-based gene synthesis. Nucleic Acids Res. 2002, 30 (10), e43.
(32) Gill, S. C.; von Hippel, P. H., Calculation of protein extinction coefficients from amino acid sequence data. Anal. Biochem. 1989, 182 (2), 319-326.
(33) Spronk, C. A. E. M.; Linge, J. P.; Hilbers, C. W.; Vuister, G. W., Improving the quality of protein structures derived by NMR spectroscopy**. J. Biomol. NMR 2002, 22 (3), 281-289.
(34) Pan, S. J.; Cheung, W. L.; Link, A. J., Engineered gene clusters for the production of the antimicrobial peptide microcin J25. Protein Expression Purif. 2010, 71 (2), 200-206.
(35) Ronquist, F.; Teslenko, M.; van der Mark, P.; Ayres, D. L.; Darling, A.; Hohna, S.; Larget, B.; Liu, L.; Suchard, M. A.; Huelsenbeck, J. P., MrBayes 3.2: Efficient Bayesian Phylogenetic Inference and Model Choice Across a Large Model Space. Syst. Biol. 2012, 61 (3), 539-542.
(36) Maddison, W. P.; Maddison, D. R., Mesquite: a modular system for evolutionary analysis. Version 3.6 2018.
(37) Allen, C. D.; Chen, M. Y.; Trick, A. Y.; Le, D. T.; Ferguson, A. L.; Link, A. J., Thermal Unthreading of the Lasso Peptides Astexin-2 and Astexin-3. ACS Chemical Biology 2016, 11 (11), 3043-3051.
(38) Maksimov, M. O.; Link, A. J., Discovery and Characterization of an Isopeptidase That Linearizes Lasso Peptides. J. Am. Chem. Soc. 2013, 135 (32), 12038-12047.
(39) Hegemann, J. D.; Zimmermann, M.; Xie, X. L.; Marahiel, M. A., Caulosegnins I-III: A Highly Diverse Group of Lasso Peptides Derived from a Single Biosynthetic Gene Cluster. J. Am. Chem. Soc. 2013, 135 (1), 210-222.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. An isolated ubonodin peptide comprising an amino acid sequence having at least 70% sequence identity to the sequence of SEQ ID NO:1, provided that the peptide does not consist of SEQ ID NO:1.

2. The ubonodin peptide of claim 1, wherein the ubonodin peptide comprises an amino acid sequence having at least 75%, at least 80%, at least 85%, at least 90% or at least 95% sequence identity to the sequence of SEQ ID NO:1.

3. The ubonodin peptide of claim 1, wherein the ubonodin peptide comprises SEQ ID NO:1.

4. The ubonodin peptide of claim 1, wherein the ubonodin peptide comprises a G28C substitution at a position corresponding to G28 in the sequence of SEQ ID NO:1.

5. The ubonodin peptide of claim 1, wherein the ubonodin peptide comprises a Y26F substitution at a position corresponding to Y26 in the sequence of SEQ ID NO:1.

6. The ubonodin peptide of claim 1, wherein the ubonodin peptide comprises a H15A substitution at a position corresponding to H15 in the sequence of SEQ ID NO:1.

7. The ubonodin peptide of claim 1, wherein the ubonodin peptide comprises a H17A substitution at a position corresponding to H17 in the sequence of SEQ ID NO:1.

8. The ubonodin peptide of claim 1, wherein the ubonodin peptide is 26 to 30 amino acids in length.

9.-10. (canceled)

11. A pharmaceutical composition, comprising an ubonodin peptide of claim 1, and a pharmaceutically acceptable carrier.

12. A method of treating a Burkholderia infection in a subject in need thereof, comprising administering to the subject an ubonodin peptide comprising an amino acid sequence having at least 70% sequence identity to the sequence of SEQ ID NO: 1.

13. The method of claim 12, wherein the Burkholderia infection is a Burkholderia thailandensis infection, Burkholderia multivorans infection, Burkholderia ubonensis infection, Burkholderia ambifaria infection, Burkholderia anthina infection, Burkholderia arboris infection, Burkholderia cenocepacia infection, Burkholderia cepacia infection, Burkholderia contaminans infection, Burkholderia diffusa infection, Burkholderia dolosa infection, Burkholderia lateens infection, Burkholderia lata infection, Burkholderia metallica infection, Burkholderia pyrrocinia infection, Burkholderia seminalis infection, Burkholderia stabilis infection, Burkholderia uronensis infection, Burkholderia vietnamiensis infection, Burkholderia mallei infection, or a combination thereof.

14. The method of claim 12, wherein the Burkholderia infection is a lung infection.

15. The method of claim 12, wherein the ubonodin peptide comprises an amino acid sequence having at least 75%, at least 80%, at least 85%, at least 90%, at least 95% or 100% sequence identity to the sequence of SEQ ID NO: 1.

16. The method of claim 12, wherein the ubonodin peptide comprises the amino acid sequence of SEQ ID NO: 1.

17. The method of claim 12, wherein the ubonodin peptide comprises a substitution selected from the group consisting of a G28C substitution in SEQ ID NO:1, a Y26F substitution in SEQ ID NO:1, a H15A substitution in SEQ ID NO:1, a H17A substitution in SEQ ID NO:1, and combinations thereof.

18. (canceled)

19. The method of claim 17, wherein the human subject has cystic fibrosis.

20.-26. (canceled)

27. A recombinant nucleic acid comprising a nucleotide sequence encoding an ubonodin peptide that comprises an amino acid sequence having at least 70% sequence identity to the sequence of SEQ ID NO: 1.

28. A recombinant nucleic acid of claim 27, comprising:
a first nucleotide sequence having at least 70% sequence identity to the sequence of SEQ ID NO: 2, wherein the first nucleotide sequence is operably linked to a first promoter;
a second nucleotide sequence having at least 70% sequence identity to the sequence of SEQ ID NO: 3;
a third nucleotide sequence having at least 70% sequence identity to the sequence of SEQ ID NO: 4;
and a fourth nucleotide sequence having at least 70% sequence identity to the sequence of SEQ ID NO: 5,
wherein the second, third and fourth nucleotide sequences are operably linked to a second promoter

29.-43. (canceled)

44. A host cell comprising the recombinant nucleic acid of claim 27.

45.-46. (canceled)

47. A method of making an ubonodin peptide, comprising:
expressing the recombinant nucleic acid of claim 28 in a host cell; and
obtaining the expressed ubonodin peptide from the host cell.
